(19) United States
(12) Patent Application Publication
(10) Pub. No.: US 2024/0302347 A1
(43) Pub. Date: Sep. 12, 2024

(54) VOLATILE BIOMARKERS FOR COLORECTAL CANCER

(71) Applicant: IMPERIAL COLLEGE INNOVATIONS LIMITED, London (GB)

(72) Inventors: George HANNA, Middlesex (GB); Georgia WOODFIELD, London (GB); Ilaria BELLUOMO, London (GB)

(21) Appl. No.: 18/550,354

(22) PCT Filed: Mar. 21, 2022
(86) PCT No.: PCT/GB2022/050701

§ 371 Date: Sep. 13, 2023

(30) Foreign Application Priority Data
Mar. 22, 2021 (GB) 2103951.6

(51) Int. Cl. G01N 33/497

(52) U.S. Cl. CPC G01N 33/4975; G01N 2800/52; G01N 2800/7028

(57) ABSTRACT

The invention relates to biomarkers, and to novel biological markers for diagnosing colorectal cancer. In particular, the invention relates to the use of these biomarkers as diagnostic and prognostic markers in assays for detecting colorectal cancer, and corresponding methods of detection. The invention also relates to methods of determining the efficacy of treating colorectal cancer with a therapeutic agent, and apparatus for carrying out the assays and methods. The assays are qualitative and/or quantitative, and are adaptable to large-scale screening and clinical trials.

### RELATED APPLICATIONS

The present application is a U.S. national phase application under 35 U.S.C. § 371 of International Application No. PCT/GB2022/050701, filed on Mar. 21, 2022 and published as WO 2022/200771 A1 on Sep. 29, 2022; which claims the priority of GB Application No. 2103951.6, filed on Mar. 22, 2021. The content of each of these related applications is incorporated herein by reference in its entirety.

The present invention relates to biomarkers, and particularly although not exclusively, so to novel biological markers for diagnosing colorectal cancer. In particular, the invention relates to the use of these biomarkers, or so-called signature compounds, as diagnostic and prognostic markers in assays for detecting colorectal cancer, and corresponding methods of detection. The invention also relates to methods of determining the efficacy of treating colorectal cancer with a therapeutic agent, and apparatus for carrying out the assays and methods. The assays are qualitative and/or quantitative, and are adaptable to large-scale screening and clinical trials.

When colorectal cancer (CRC) is diagnosed at its earliest stage, more than 9 in 10 people with CRC will survive their disease for five years or more, compared with less than 1 in 10 when diagnosed at the latest disease stage [1]. The utilization of bowel symptoms as the primary diagnostic basis for CRC has been shown to have a very poor positive predictive value [2]. Risk of CRC in symptomatic patients can be assessed by different investigations. Colonoscopy is the gold standard investigation but the large scale of its application has resource implications and its cost-effectiveness depends on the predictive values of different symptoms. Guaiac faecal occult blood test has good sensitivity of 87-98% in CRC detection, but highly variable and often unsatisfactory specificity (13-79%), requiring the repetition of the test on multiple stool samples. To date, the faecal occult blood test is neither recommended nor available for use as an intermediate test [3-6]. The faecal immunochemical test requires a single stool sample. Four systems are fully automated, and provide a quantitative measure of haemoglobin, allowing selection of a threshold of positivity to fit specific circumstances. As a result, the research data available on sensitivity and specificity for CRC is based on small numbers of cancers. The data suggest that, depending on the selected threshold for positivity, the sensitivity for CRC varies between 35% and 86% with specificity between 85% and 95% [5,6]. However, there are no data on the sensitivity of the newer quantitative test for early-stage cancers. The multi-target stool DNA test, when compared with the faecal immunochemical test in a large multicentre study, showed a better specificity (92 vs. 73%), but a lower sensitivity (90 vs. 96%) [7].

An alternative approach to faecal-based tests is exhaled breath testing with the potential for high compliance because of the nature of the test and the possibility for testing more than one disease with different volatile organic compounds (VOC) discriminative signatures [8,9]. Researchers using gas chromatography mass spectrometry (GC-MS) have suggested the existence of a breath VOC profile specific to CRC [10]. GC-MS is a good technique for VOC identification, however it is semi-so quantitative in nature, and thus limited in the ability of research findings to be reproduced by different research groups. Furthermore, there is a substantial analytical time for each sample, which does not naturally lend itself to high throughput analysis.

Selected ion flow tube mass spectrometry (SIFT-MS) has the advantage of being quantitative and permits real-time analysis [11,12].

Accordingly, what is required is a reliable non-invasive marker to identify patients suffering from colorectal cancer. A diagnostic method to identify those patients with colorectal cancer would be of immense benefit to patients and would raise the possibility of early treatment and improved prognosis.

The inventors have now determined several biomarkers or so-called signature compounds as being indicative (diagnostically and prognostically) of colorectal cancer.

As described in the Examples, patients were recruited and split into two separate groups, CRC patients and non-CRC patients (i.e. the control group). The control group included patients with a colonoscopy diagnosis of normal, benign pathology, inflammatory bowel disease, low risk polyp(s), intermediate risk polyp(s), or high risk polyp(s). Breath was collected from patients using the ReCIVA system and analysis was performed using GC-MS. Of the signature volatile organic compounds (VOCs) identified, 15 were statistically significantly different between CRC and non-CRC patients, including dimethyl sulphide, phenol, and compounds from the ester, alcohol, alkane and non-aromatic cyclic hydrocarbon chemical classes. The inventors demonstrated that analysis of VOCs could robustly predict the presence of CRC from positive and negative controls using the breath, with an area under the receiver operating characteristic (ROC) curve of 0.87, a sensitivity of 77%, a specificity of 87%, and a negative predictive value of 97%. Using just 15 VOCs, CRC could be detected from controls with an area under the ROC curve of 0.83.

Hence, in a first aspect of the invention, there is provided a method for diagnosing a subject suffering from colorectal cancer, or a pre-disposition thereto, or for providing a prognosis of the subject's condition, the method comprising analysing the concentration of a signature compound in a bodily sample from a test subject and comparing this concentration with a reference for the concentration of the signature compound in an individual who does not suffer from colorectal cancer, wherein:
(i) an increase in the concentration of a signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, or
(ii) a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the subject is suffering from colorectal cancer, or has a pre-disposition thereto, or provides a negative prognosis of the subject's condition, wherein formulae (I), (II) and (III) are:

R¹-L¹-OH (I)

R²SR³ (II)

R⁴-L²-L³-OH (III),

wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

In a second aspect, there is provided a method for determining the efficacy of treating a subject suffering from colorectal cancer with a therapeutic agent or a specialised diet, the method comprising analysing the concentration of a signature compound in a bodily sample from a test subject and comparing this concentration with a reference for the concentration of the signature compound in a sample taken from the subject at an earlier time point, wherein:
(i) a decrease in the concentration of the signature compound selected from a C₁₋₁₂ ester, a C₃₋₂₀ cycloalkane, a C₃₋₂₀ cycloalkene, an alcohol of formula (I), a sulphide of so formula (II), or an analogue or derivative thereof, in the bodily sample from the test subject, compared to the reference, or (ii) an increase in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the treatment regime with the therapeutic agent or the specialised diet is effective, or wherein (i) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or (ii) a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the treatment regime with the therapeutic agent or the specialised diet is ineffective, wherein formulae (I), (II) and (III) are:

R¹-L-OH (I)

R²SR³ (II)

R⁴-L²-L³-OH (III)

wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

In a third aspect, there is provided an apparatus for diagnosing a subject suffering from colorectal cancer, or a pre-disposition thereto, or for providing a prognosis of the subject's condition, the apparatus comprising:—
(i) means for determining the concentration of a signature compound in a sample from a test subject; and
(ii) a reference for the concentration of the signature compound in a sample from an individual who does not suffer from colorectal cancer, wherein the apparatus is used to identify: (i) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, or (ii) a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, thereby suggesting that the subject suffers from colorectal cancer, or has a pre-disposition thereto, or provides a negative prognosis of the subject's condition, wherein formulae (I), (II) and (III) are:

R¹-L¹-OH (I)

R²SR³ (II)

R⁴-L²-L³-OH (III),

wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

In a fourth aspect, the invention provides an apparatus for determining the efficacy of treating a subject suffering from colorectal cancer with a therapeutic agent or a specialised diet, the apparatus comprising:—
(a) means for determining the concentration of a signature compound in a sample from a test subject; and
(b) a reference for the concentration of the signature compound in a sample taken from the subject at an earlier time point,
   wherein the apparatus is used to identify:
(i) a decrease in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or an increase in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, thereby suggesting that the treatment regime with the therapeutic agent or the specialised diet is effective; or
(ii) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, thereby suggesting that the treatment regime with the therapeutic agent or the specialised diet is ineffective, wherein formulae (I), (II) and (III) are:

R¹-L¹-OH (I)

R²SR³ (II)

R⁴-L²-L³-OH (III),

wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

According to a fifth aspect of the invention, there is provided a method of treating an individual suffering from colorectal cancer, said method comprising the steps of:
(i) determining the concentration of a signature compound in a sample from a test subject concentration, wherein (i) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, or (ii) a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the subject is suffering from colorectal cancer, or has a pre-disposition thereto, or has a negative prognosis, wherein formulae (I), (II) and (III) are:

R¹-L¹-OH (I)

R²SR³ (II)

R⁴-L²-L³-OH (III)

wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl; and
(ii) administering, or having administered, to the test subject, a therapeutic agent or putting the test subject on a specialised diet, wherein the therapeutic agent or the specialised diet prevents, reduces or delays progression of colorectal cancer.

In a sixth aspect, there is provided use of a signature compound selected from the group consisting of a C₁₋₁₂ ester, a C₃₋₂₀ cycloalkane, a C₃₋₂₀ cycloalkene, a C₁₋₂₀ alkane, a C₂₋₂₀ alkene, a C₂₋₂₀ alkyne, an alcohol of formula (I), a sulphide of formula (II), and an alcohol of formula (III), or an analogue or derivative thereof, as a biomarker for diagnosing a subject suffering from colorectal cancer, or a pre-disposition thereto, or for providing a prognosis of the subject's condition, wherein formulae (I), (II) and (III) are:

R¹-L-OH (I)

R²SR³ (II)

R⁴-L²-L³-OH (III),

wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

The expression “determining the concentration” can include either determining the relative abundance or level of signature compound in the sample, which are semi-quantitative given by peak area, or determining the actual quantity of signature compound. As described in the Examples, the inventors have surprisingly demonstrated that an increase in the concentration of propyl propionate, allyl acetate, methyl 2-butynoate, 1,3-Dioxolane-2-methanol, 2,2,4-Trimethyl-3-pentanol, cyclopropane, 3,4-dimethyl-1,5-Cyclooctadiene, or dimethyl sulphide, is indicative of colorectal cancer. Additionally, the inventors have surprisingly shown that a decrease in the concentration of 2-Phenoxy-ethanol, 1-undecanol, phenol, or 3-ethyl-hexane, is indicative of colorectal cancer. The methods, apparatus and uses described herein may also comprise analysing the concentration, abundance or level of an analogue or a derivative of the signature compounds described herein. Examples of suitable analogues or derivatives of chemical groups which may be assayed include alcohols, ketones, aromatics, organic acids and gases (such as CO, CO₂, NO, NO₂, H₂S, SO₂, and CH₄).

In an embodiment in which the signature compound is a C₁-C₁₂ ester, preferably the compound is a C₃₋₈ ester, and most preferably a C₅₋₆ ester.

The ester may be an ester of formula IV:

R⁶C(O)OR⁷ (IV),

wherein R⁶ and R⁷ are independently a C₁₋₆ alkyl, a C₂₋₆ alkenyl or a C₂₋₆ alkynyl.

In some embodiments, R⁶ and R⁷ are independently a C₁₋₄ alkyl, a C₂₋₄ alkenyl or a C₂₋₄ alkynyl. More preferably, R⁶ and R⁷ are independently a C₁₋₃ alkyl, a C₂₋₃ alkenyl or a C₂₋₃ alkynyl. R⁶ and R⁷ may independently be methyl, ethyl, propyl, ethenyl, propenyl, ethynyl or propynyl. Most preferably, R⁶ is methyl, ethyl or 1-propynyl. Most preferably, R⁷ is methyl, n-propanyl or 2-propenyl.

In a preferred embodiment, the C₁-C₁₂ ester is propyl propionate, allyl acetate or methyl 2-butynoate.

In an embodiment in which the signature compound is a C₃₋₂₀ cycloalkane or a C₃₋₂₀ cycloalkene, preferably the compound is a C₃₋₁₅ cycloalkane or a C₃₋₁₅ cycloalkene, more preferably a C₃₋₁₀ cycloalkane or a C₃₋₁₀ cycloalkene. In some embodiments, the compound may be a C₃₋₆ cycloalkane, more preferably a C₃₋₄ cycloalkane. In some embodiments, the compound may be a C₅₋₁₀ cycloalkene, more preferably a C₈₋₁₀ cycloalkene.

Preferably, the C₃₋₂₀ cycloalkane or C₃₋₂₀ cycloalkene is cyclopropane, or 3,4-dimethyl-1,5-cyclooctadiene.

In an embodiment in which the signature compound is a C₁₋₂₀ alkane, a C₂₋₂₀ alkene, or a C₂₋₂₀ alkyne, preferably the compound is a C₄₋₁₂ alkane, a C₄₋₁₂ alkene or a C₄₋₁₂ alkyne, more preferably a C₆₋₁₀ alkane, a C₆₋₁₀ alkene or a C₆₋₁₀ alkyne, even more preferably a C₇₋₉ alkane, a C₇₋₉ alkene or a C₇₋₉ alkyne, and most preferably a C₈ alkane. The alkane, alkene or alkyne is preferably a branched chain alkane, alkene or alkyne.

In a preferred embodiment, the C₁₋₂₀ alkane, C₂₋₂₀ alkene, or C₂₋₂₀ alkyne is 3-ethyl-hexane.

In an embodiment in which the signature compound is an alcohol of formula I:

R¹-L-OH (I),

preferably R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl; and
L¹ is absent or a C₁₋₆ alkylene, a C₂₋₆ alkenylene or a C₂₋₆ alkynylene.
L¹ may be absent or a C₁₋₃ alkylene, a C₂₋₃ alkenylene or a C₂₋₃ alkynylene. Preferably, L¹ is absent or methylene.
R¹ may be a C₃₋₁₂ cycloalkyl or a 3 to 12 membered heterocycle. More preferably, R¹ is a C₅₋₆ cycloalkyl or a 5 to 6 membered heterocycle. Most preferably, R¹ is a 5 membered heterocycle. R¹ may be 1,3-dioxolanyl.

In alternative embodiments, L¹ is absent and R¹ is a C₃₋₁₈ alkyl, a C₃₋₁₈ alkenyl or a C₃₋₁₈ alkynyl. R¹ may be a C₄₋₁₅ alkyl, a C₄₋₁₅ alkenyl or a C₄₋₁₅ alkynyl. More preferably, R¹ is a C₆₋₁₀ alkyl, a C₆₋₁₂ alkenyl or a C₆₋₁₀ alkynyl, and most preferably a C₇₋₉ alkyl, a C₆₋₉ alkenyl or a C₆₋₉ alkynyl. The alkyl, alkenyl or alkynyl is preferably a branched chain alkyl, alkenyl or alkynyl. R¹ may be 2,2,4-trimethyl-3-pentanyl.

In a preferred embodiment, the alcohol of formula (I) is 1,3-dioxolane-2-methanol or 2,2,4-trimethyl-3-pentanol.

In an embodiment in which the signature compound is an alcohol of formula III:

R⁴-L²-L³-OH (III),
preferably R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.
L² may be absent or O.
L³ may be absent or a C₁₋₃ alkylene, a C₂₋₃ alkenylene or a C₂₋₃ alkynylene. Preferably, L³ is absent, methylene or ethylene. Most preferably, L³ is absent or ethylene.
R⁴ may be a C₆₋₁₂ aryl or a 5 to 12 membered heteroaryl. More preferably, R⁴ is a phenyl or a 5 to 6 membered heteroaryl. Most preferably, R⁴ is phenyl.
In alternative embodiments, L² and L³ are absent and R³ is a C₃₋₁₈ alkyl, a C₃₋₁₈ alkenyl or a C₃₋₁₈ alkynyl. R³ may be a C₅₋₁₇ alkyl, a C₅₋₁₇ alkenyl or a C₅₋₁₇ alkynyl. More preferably, R³ is a C₇₋₁₄ alkyl, a C₇₋₁₄ alkenyl or a C₇₋₁₄ alkynyl, and most preferably a C₁₀₋₁₂ alkyl, a C₁₀₋₁₂ alkenyl or a C₁₀₋₁₂ alkynyl. Preferably, the alkyl, alkenyl or alkynyl is a straight chain alkyl, alkenyl or alkynyl. R³ may be 1-undecanyl.

In a preferred embodiment, the alcohol of formula (III) is 2-phenoxy-ethanol, 1-undecanol or phenol. Most preferably, the alcohol of formula (III) is phenol.

In an embodiment in which the signature compound is a sulphide of formula (II):

R²SR³ (II),
preferably R² and R³ are independently a C₁₋₆ alkyl, a C₂₋₆ alkenyl or a C₂₋₆ alkynyl.

Preferably R² and R³ are independently a C₁₋₃ alkyl, a C₂₋₃ alkenyl or a C₂₋₃ alkynyl. Most preferably R² and R³ are both methyl.

In a preferred embodiment, the sulphide is dimethyl sulphide.

In an alternative embodiment, the signature compound may be defined by its retention time. Retention time is a measure of the time a compound spends in a chromatographic column, and is dependent upon its volatility and affinity for the column. More volatile compounds will have a lower retention time, while less volatile compounds will have a higher retention time.

In an embodiment in which the signature compound is a C₁-C₁₂ ester, preferably the compound has a retention time of 20-26 minutes, more preferably 21-25 minutes, and more preferably 22-24 minutes. Most preferably, the compound has a retention time of 22.02, 22.24, or 23.53 minutes. Alternatively, the compound has a retention time of 30-35 minutes, more preferably 31-34 minutes, and more preferably 32-33 minutes. Most preferably, the compound has a retention time of 32.69 minutes.

In an embodiment in which the signature compound is a C₃₋₂₀ cycloalkane or a C₃₋₂₀ cycloalkene, preferably the compound has a retention time of 2-7 minutes, more preferably 3-6 minutes, and more preferably 4-5 minutes. Most preferably, the compound has a retention time of 4.75 minutes. Alternatively, the compound has a retention time of 29-34 minutes, more preferably 30-33 minutes, and more preferably 31-32 minutes. Most preferably, the compound has a retention time of 31.14 minutes.

In an embodiment in which the signature compound is an alcohol of formula (I), preferably the compound has a retention time of 4-9 minutes, more preferably 5-8 minutes, and more preferably 6-7 minutes. Most preferably, the compound has a retention time of 6.68 minutes. Alternatively, the compound has a retention time of 29-34 minutes, more preferably 30-33 minutes, and more preferably 31-32 minutes. Most preferably, the compound has a retention time of 31.71 minutes.

In an embodiment in which the signature compound is a sulphide of formula (II), preferably the compound has a retention time of 7-12 minutes, more preferably 8-11 so minutes, and more preferably 9-10 minutes. Most preferably, the compound has a retention time of 9.27 minutes.

In an embodiment in which the signature compound is a C₁₋₂₀ alkane, a C₂₋₂₀ alkene, or a C₂₋₂₀ alkyne, preferably the compound has a retention time of 19-24 minutes, more preferably 20-23 minutes, and more preferably 21-22 minutes. Most preferably, the compound has a retention time of 21.26 minutes. Alternatively, the compound has a retention time of 37-42 minutes, more preferably 38-39 minutes, or 40-41 minutes. Most preferably, the compound has a retention time of 38.74 minutes, or 40.12 minutes.

In an embodiment in which the signature compound is an alcohol of formula (III), preferably the compound has a retention time of 16-21 minutes, more preferably 17-20 minutes, and more preferably 18-19 minutes. Most preferably, the compound has a retention time of 18.11 minutes. Alternatively, the compound has a retention time of 22-27 minutes, more preferably 23-26 minutes, and more preferably 24-25 minutes. Most preferably, the compound has a retention time of 24.65 minutes. Alternatively, the compound has a retention time of 38-43 minutes, more preferably 39-42 minutes, and more preferably 40-41 minutes. Most preferably, the compound has a retention time of 40.52 minutes.

Thus, in a most preferred embodiment, the first aspect comprises a method for diagnosing a subject suffering from colorectal cancer, or a pre-disposition thereto, or for providing a prognosis of the subject's condition, the method comprising analysing the concentration of a signature compound in a bodily sample from a test subject and comparing this concentration with a reference for the concentration of the signature compound in an individual who does not suffer from colorectal cancer, wherein (i) an increase in the concentration of the signature compound selected from propyl propionate, allyl acetate, methyl 2-butynoate, 1,3-Dioxolane-2-methanol, 2,2,4-Trimethyl-3-pentanol, cyclopropane, 3,4-dimethyl-1,5-Cyclooctadiene, or dimethyl sulphide, or an analogue or derivative thereof, in the bodily sample from the test subject, or (ii) a decrease in the concentration of the signature compound selected from 2-Phenoxy-ethanol, 1-undecanol, phenol, or 3-ethyl-hexane, or an analogue or derivative thereof, in the bodily sample from the test subject, compared to the reference, suggests that the subject is suffering from colorectal cancer, or has a pre-disposition thereto, or provides a negative prognosis of the subject's condition.

It will be appreciated that, in their most preferred embodiments, the aspects involve detecting an increase and/or decrease of the same signature compounds as defined in the previous paragraph.

An important feature of any useful biomarker used in disease diagnosis and prognosis is that it exhibits high sensitivity and specificity for a given disease. As explained in the examples, the inventors have surprisingly demonstrated that a number of signature compounds found in the exhaled breath from test subjects serve as robust biomarkers for colorectal cancer, and can therefore be used for the detection and prognosis of this disease. In addition, the inventors have shown that using such signature compounds as a biomarker for disease employs an assay which is simple, reproducible, non-invasive and inexpensive, and with minimal inconvenience to the patient.

Advantageously, the methods and apparatus of the invention provide a non-invasive means for diagnosing colorectal cancer. The method according to the first aspect is useful for enabling a clinician to make decisions with regards to the best course of treatment for a subject who is currently suffering, or who may suffer, from colorectal cancer. It is preferred that the method of the first aspect is useful for enabling a clinician to decide how to treat a subject who is currently suffering from colorectal cancer. In addition, the methods of the first and second aspects are useful for monitoring the efficacy of a putative treatment for the colorectal cancer. For example, treatment may comprise administration of chemotherapy, chemoradiotherapy with or without surgery, or endoscopic resection.

Hence, the apparatus according to the third and fourth aspects are useful for providing a prognosis of the subject's condition, such that the clinician can carry out the treatment according to the fifth aspect. The apparatus of the third aspect may be used to monitor the efficacy of a putative treatment for the colorectal cancer. The methods and apparatus are therefore very useful for guiding a treatment regime for the clinician, and to monitor the efficacy of such a treatment regime. The clinician may use the apparatus of the invention in conjunction with existing diagnostic tests to improve the accuracy of diagnosis.

The subject may be any animal of veterinary interest, for instance, a cat, dog, horse etc. However, it is preferred that the subject is a mammal, such as a human, either male or female.

Preferably, a sample is taken from the subject, and the concentration of the signature compound in the bodily sample is then measured.

The signature compounds, which are detected, may be known as volatile organic compounds (VOCs), which lead to a fermentation profile, and they may be detected in the bodily sample by a variety of techniques. In one embodiment, these compounds may be detected within a liquid or semi-solid sample in which they are dissolved. In a preferred embodiment, however, the compounds are detected from gases or vapours. For example, as the signature compounds are VOCs, they may emanate from, or from part of, the sample, and may thus be detected in gaseous or vapour form.

The apparatus of the third or fourth aspect may comprise sample extraction means for obtaining the sample from the test subject. The sample extraction means may comprise a needle or syringe or the like. The apparatus may comprise a sample collection container for receiving the extracted sample, which may be liquid, gaseous or semi-solid.

Preferably, the sample is any bodily sample into which the signature compound is present or secreted. For example, the sample may comprise urine, faeces, hair, sweat, saliva, blood or tears. The inventors believe that the VOCs are breakdown products of other compounds found within the blood. In one embodiment, blood samples may be assayed for the signature compound's levels immediately. Alternatively, the blood may be stored at low temperatures, for example in a fridge or even frozen before the concentration of signature compound is determined. Measurement of the signature compound in the bodily sample may be made on whole blood or processed blood.

In other embodiments, the sample may be a urine sample. It is preferred that the concentration of the signature compound in the bodily sample is measured in vitro from a urine sample taken from the subject. The compound may be detected from gases or vapours emanating from the urine sample. It will be appreciated that detection of the compound in the gas phase emitted from urine is preferred.

It will also be appreciated that “fresh” bodily samples may be analysed immediately after they have been taken from a subject. Alternatively, the samples may be frozen and stored. The sample may then be de-frosted and analysed at a later date.

Most preferably, however, the bodily sample may be a breath sample from the test subject. The sample may be collected by the subject performing exhalation through the mouth and/or nose, preferably after nasal inhalation. Preferably, the sample comprises the subject's alveolar air. Preferably, the alveolar air was collected over dead space air by capturing end-expiratory breath. VOCs from breath bags were then preferably pre-concentrated onto thermal desorption tubes by transferring breath across the tubes.

Accordingly, in a preferred embodiment, the concentration of the signature compound selected from a C₁₋₁₂ ester, a C₃₋₂₀ cycloalkane, a C₃₋₂₀ cycloalkene, an alcohol of formula (I), a sulphide of formula (II), a C₁₋₂₀ alkane, a C₂₋₂₀ alkene, a C₂₋₂₀ alkyne, and an alcohol of formula (III), or an analogue or derivative thereof, is analysed in a breath sample. In some embodiments, the concentration of the signature compound selected from propyl propionate, allyl acetate, methyl 2-butynoate, 1,3-Dioxolane-2-methanol, 2,2,4-Trimethyl-3-pentanol, cyclopropane, 3,4-dimethyl-1,5-Cyclooctadiene, dimethyl sulphide, 2-Phenoxy-ethanol, 1-undecanol, phenol, or 3-ethyl-hexane, or an analogue or derivative thereof, is analysed in a breath sample. Preferably, the concentration of 3-ethyl-hexane is analysed in a breath sample.

The difference in concentration of signature compound in the methods of the first aspect or the apparatus of the third aspect may be an increase or a decrease compared to the reference. As described in the examples, the inventors monitored the concentration of the signature compounds in numerous patients who suffered from colorectal cancer, and compared them to the concentration of these same compounds in individuals who did not suffer from colorectal cancer (i.e. reference or controls).

They demonstrated that there was a statistically significant increase or decrease in the concentration of these compounds in the patients suffering from colorectal cancer.

It will be appreciated that the concentration of signature compound in patients suffering from colorectal cancer is highly dependent on a number of factors, for example how far the cancer has progressed, and the age and gender of the subject. It will also be appreciated that the reference concentration of signature compound in individuals who do not suffer from colorectal cancer may fluctuate to some degree, but that on average over a given period of time, the concentration tends to be substantially constant. In addition, it should be appreciated that the concentration of signature compound in one group of individuals who suffer from colorectal cancer may be different to the concentration of that compound in another group of individuals who do not suffer from colorectal cancer. However, it is possible to determine the average concentration of signature compound in individuals who do not suffer from the cancer, and this is referred to as the reference or ‘normal’ concentration of signature compound. The normal concentration corresponds to the reference values discussed above.

In one embodiment, the methods of the invention preferably comprise determining the ratio of chemicals within the sample, such as a breath sample (i.e. using other components within it as a reference), and then compare these markers to the disease to show if they are elevated or reduced.

The signature compound is preferably a volatile organic compound (VOC), which leads to a fermentation profile, and it may be detected in or from the bodily sample by a variety of techniques. Thus, these compounds may be detected using a gas analyser. Examples of suitable detector for detecting the signature compound preferably includes an electrochemical sensor, a semiconducting metal oxide sensor, a quartz crystal microbalance sensor, an optical dye sensor, a fluorescence sensor, a conducting polymer sensor, a composite polymer sensor, or optical spectrometry.

The inventors have demonstrated that the signature compounds can be reliably detected using GC-MS or GC-TOF. Dedicated sensors could be used for the detection step.

The reference values may be obtained by assaying a statistically significant number of control samples (i.e. samples from subjects who do not suffer from colorectal cancer). Accordingly, the reference (ii) according to the apparatus of the third or fourth aspects of the invention may be a control sample (for assaying).

The apparatus preferably comprises a positive control (most preferably provided in a container), which corresponds to the signature compound(s). The apparatus preferably comprises a negative control (preferably provided in a container). In a preferred embodiment, the apparatus may comprise the reference, a positive control and a negative control. The apparatus may also comprise further controls, as necessary, such as “spike-in” controls to provide a reference for concentration, and further positive controls for each of the signature compounds, or an analogue or derivative thereof.

Accordingly, the inventors have realised that the difference in concentrations of the signature compound between the reference normal (i.e. control) and increased/decreased levels, can be used as a physiological marker, suggestive of the presence of colorectal cancer in the test subject. It will be appreciated that if a subject has an increased/decrease concentration of one or more signature compounds which is considerably higher/lower than the ‘normal’ concentration of that compound in the reference, control value, then they would be at a higher risk of having the cancer, or a condition that was more advanced, than if the concentration of that compound was only marginally higher/lower than the ‘normal’ concentration.

The inventors have noted that the concentration of signature compounds referred to herein in the test individuals was statistically more than the reference concentration (as calculated using the method described in the Example). This may be referred to herein as the ‘increased’ concentration of the signature compound.

The skilled technician will appreciate how to measure the concentrations of the signature compound in a statistically significant number of control individuals, and the concentration of compound in the test subject, and then use these respective figures to determine whether the test subject has a statistically significant increase/decrease in the compound's concentration, and therefore infer whether that subject is suffering from colorectal cancer.

In the method of the second aspect and the apparatus of the fourth aspect, the difference in the concentration of the signature compound in the bodily sample compared to the corresponding concentration in the reference is indicative of the efficacy of treating the subject's colorectal cancer with the therapeutic agent, and surgical resection. The difference may be an increase or a decrease in the concentration of the signature compound in the bodily sample compared to the reference value. In this embodiment, the reference sample is a sample taken from the subject at an earlier time point. The reference sample may have been taken from the subject prior to commencing treatment. Accordingly, the method and/or apparatus may show if an improvement has occurred in the subject since the start of treatment.

Alternatively, or additionally, the reference sample may comprise a sample taken from the subject subsequent to commencing treatment. In some embodiments, the reference sample may comprise a plurality of samples taken from the subject at different time points subsequent to commencing treatment. For example, the plurality of samples may be one or more days apart, one or more weeks apart, one or more months apart, or even one or more years apart. For example, samples may be taken from the subject at least once, twice or three times every week, every month or every year. The samples may be taken at evenly spaced intervals or a randomly spaced intervals. The plurality of samples may also include a sample taken from the subject prior to commencing treatment, or after treatment has started. Accordingly, the method of the second aspect and the apparatus of the fourth aspect can determine if an improvement is ongoing.

In embodiments where the concentration of the compound in the bodily sample is lower than the corresponding concentration in the reference, then this would indicate that the therapeutic agent is successfully treating the cancer in the test subject. This would apply to a signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof.

Conversely, where the concentration of the signature compound in the bodily sample is higher than the corresponding concentration in the reference, then this would indicate that the therapeutic agent is not successfully treating the cancer. This would apply to a signature compound selected from a C₁₋₂₀ alkane, a C₂₋₂₀ alkene, a C₂₋₂₀ alkyne, and an alcohol of formula (III), or an analogue or derivative thereof.

In another aspect, there is provided a method for determining the efficacy of treating a subject suffering from colorectal cancer with a therapeutic agent or a specialised diet, the method comprising analysing the concentration of a signature compound in a bodily sample from a test subject and comparing this concentration with a reference for the concentration of the signature compound in an individual who does not suffer from colorectal cancer, wherein:
(i) a decrease in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or (ii) an increase in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the treatment regime with the therapeutic agent or the specialised diet is effective, or wherein (i) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or (ii) a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the treatment regime with the therapeutic agent or the specialised diet is ineffective, wherein formulae (I), (II) and (III) are:

R¹-L¹-OH (I)

R²SR³ (II)

R⁴-L²-L³-OH (III),

wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

In another aspect, the invention provides an apparatus for determining the efficacy of treating a subject suffering from colorectal cancer with a therapeutic agent or a specialised diet, the apparatus comprising:—
(a) means for determining the concentration of a signature compound in a sample from a test subject; and
(b) a reference for the concentration of the signature compound in a sample from an individual who does not suffer from colorectal cancer,
   wherein the apparatus is used to identify:
(i) a decrease in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or an increase in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, thereby suggesting that the treatment regime with the therapeutic agent or the specialised diet is effective; or
(ii) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, thereby suggesting that the treatment regime with the therapeutic agent or the specialised diet is ineffective, wherein formulae (I), (II) and (III) are:

R¹-L¹-OH (I)

R²SR³ (II)

R⁴-L²-L³-OH (III),
wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

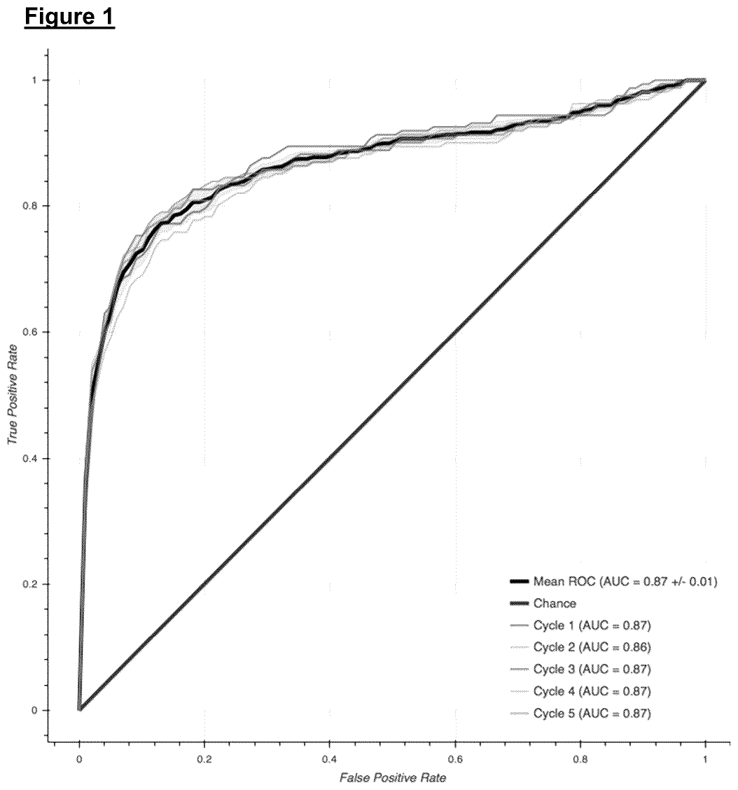

FIG. 1 shows the receiver operating characteristic (ROC) curve for the prediction of CRC using all of the detected VOCs from CRC patients (n=162) and non-CRC patients (n=1270). The area under the ROC is 0.87.

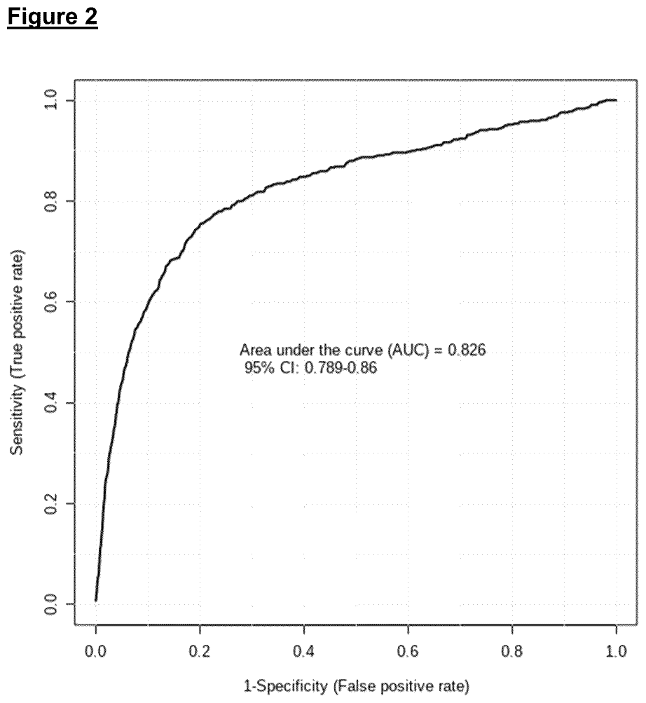

FIG. 2 shows the ROC curve illustrating the predictive power of the 15 significant VOCs in determining CRC patients from non-CRC patients, with an area under the curve of 0.83.

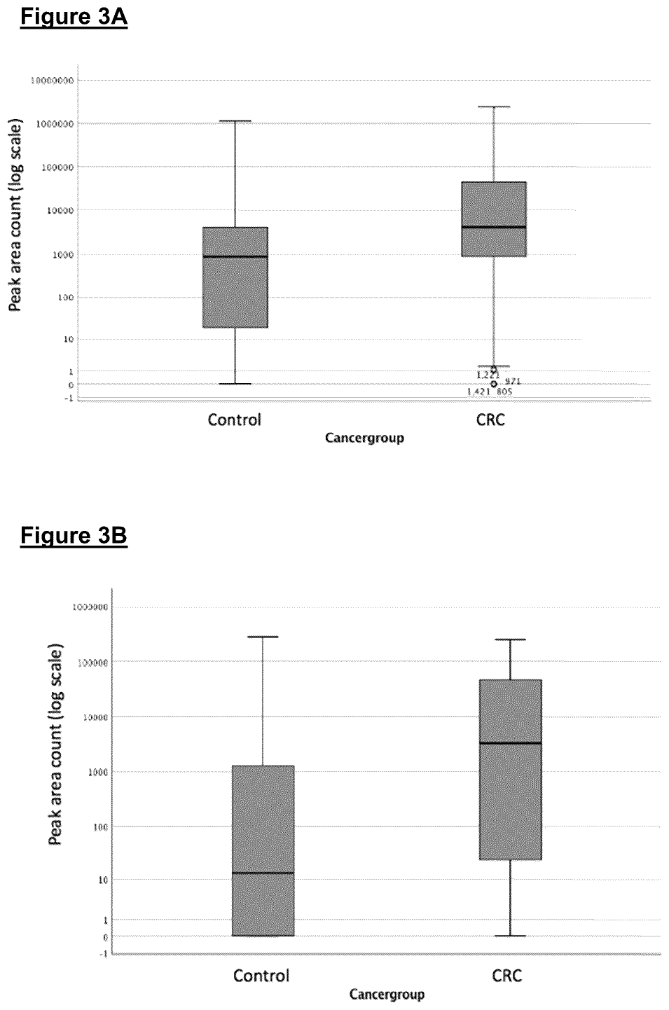

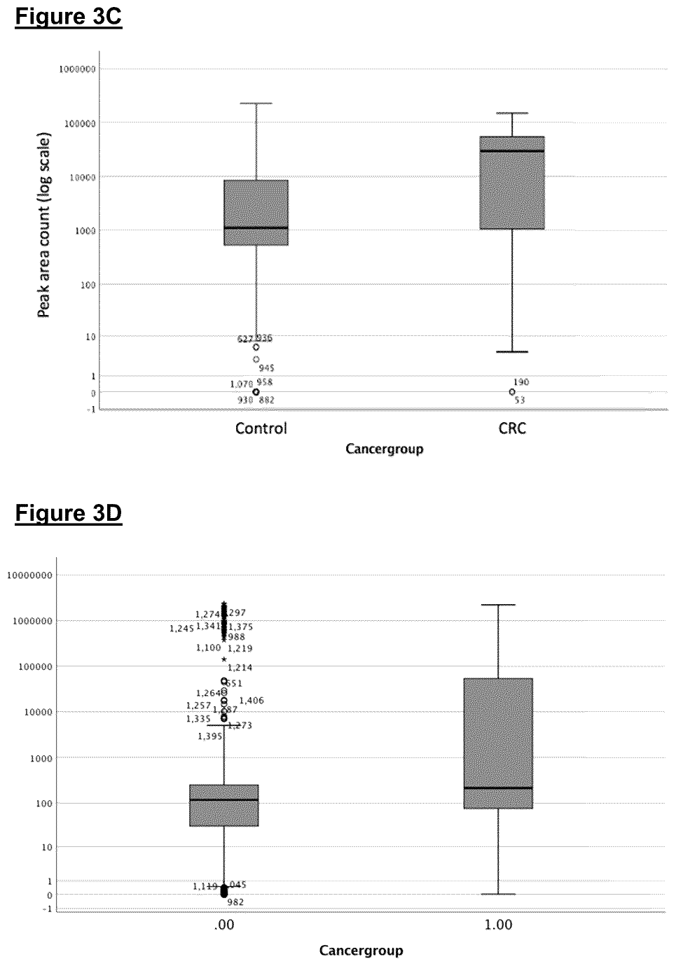

FIGS. 3A-3D show the abundance of four esters in the breath of non-CRC vs CRC patients. All four esters, propyl propionate (VOC 1, FIG. 3A), allyl acetate (VOC 8, FIG. 3B), an overlapping ester to allyl acetate (VOC 9, FIG. 3C), and methyl 2-butynoate (VOC 12, FIG. 3D), showed higher abundance in the breath of patients with CRC compared to those without CRC. The median is represented by the solid horizontal line, the whiskers represent the minimal and maximal value, and the box represents the interquartile range.

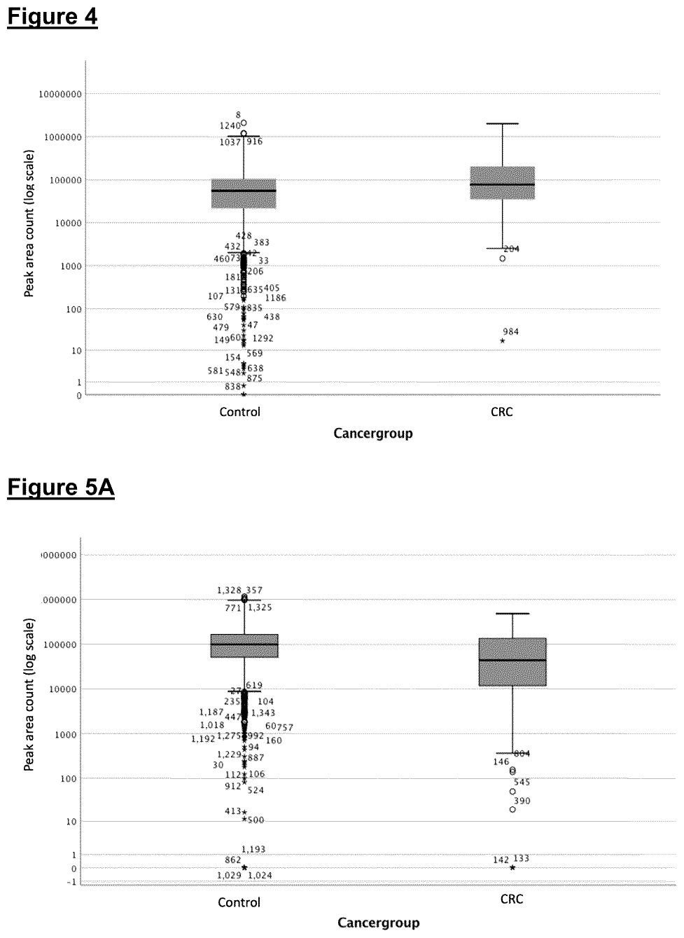

FIG. 4 shows that the abundance of dimethyl sulphide in the breath was significantly higher in patients with CRC compared to those without CRC. The median is represented by the solid horizontal line, the whiskers represent the minimal and maximal value, and the box represents the interquartile range.

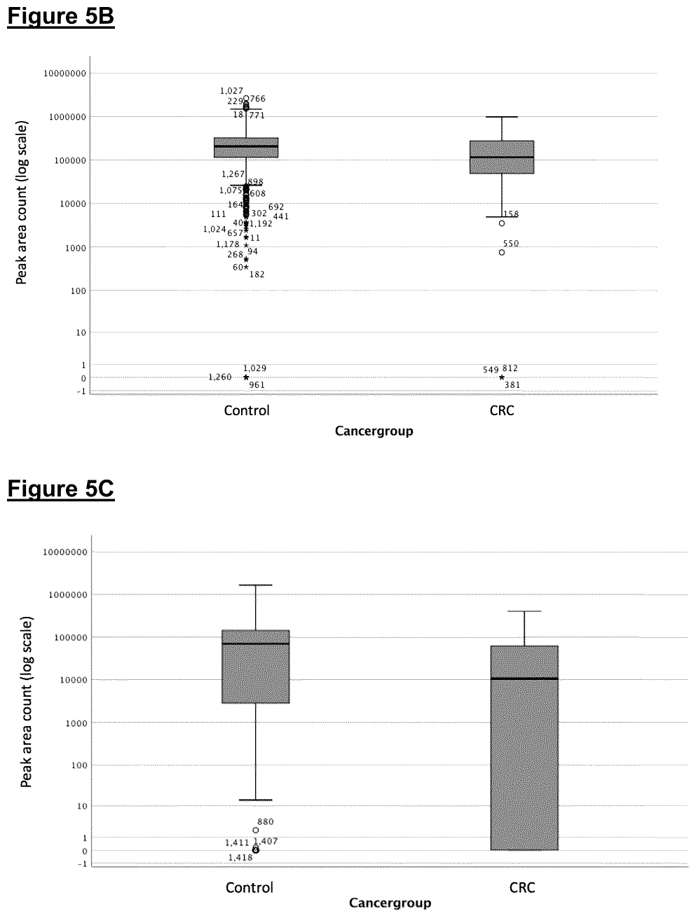

FIGS. 5A-5C show the abundance of three alkanes in the breath of non-CRC vs CRC patients. Alkane (VOC 3, FIG. 5A), alkane (VOC 11, FIG. 5B), and 3-ethyl-hexane (VOC 15, FIG. 5C), were all present in a significantly lower abundance in the breath of patients with CRC compared to those without CRC. The median is represented by the solid horizontal line, the whiskers represent the minimal and maximal value, and the box represents the interquartile range.

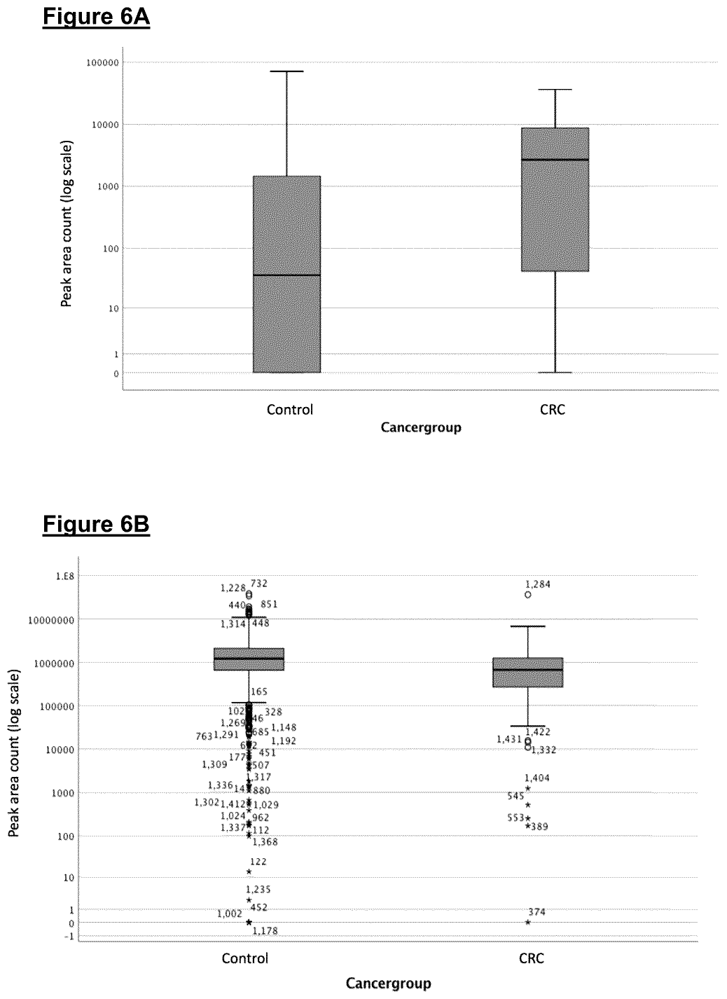

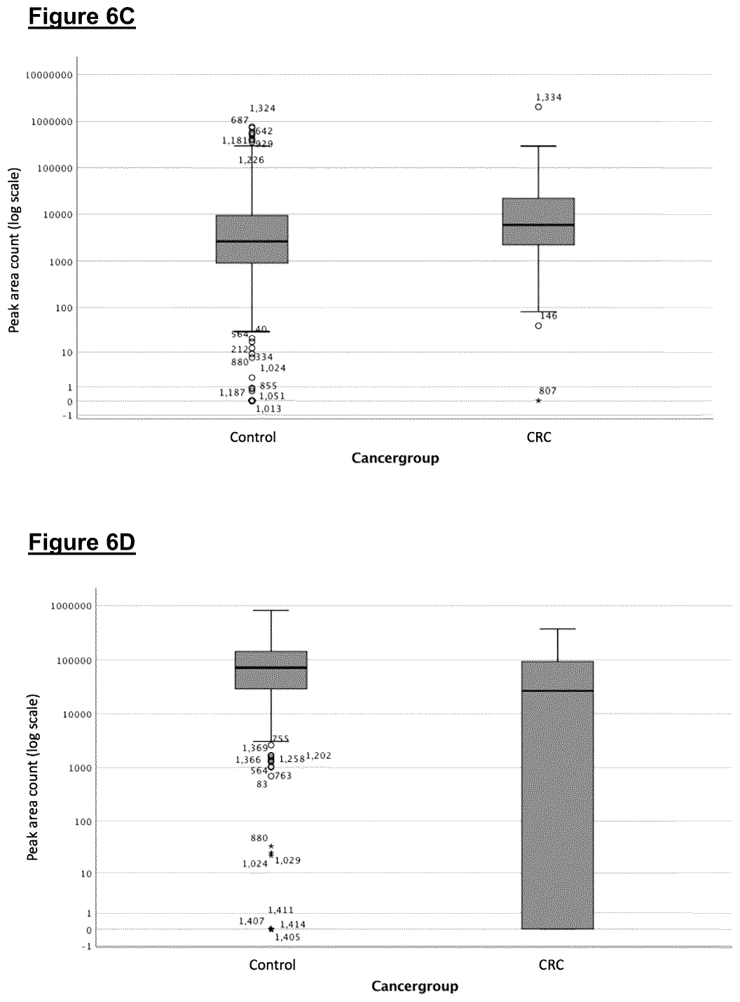

FIGS. 6A-6D show the abundance of four alcohols in the breath of non-CRC vs CRC patients. 1,3-Dioxolane-2-methanol (VOC 4, FIG. 6A) and 2,2,4-trimethyl-3-pentanol (VOC 10, FIG. 6C), were found to be present in significantly higher abundance in the breath of patients with CRC compared to those without CRC. 2-phenoxy-ethanol (VOC 5, FIG. 6B) and 1-undecanol (VOC 13, FIG. 6D) were found to be present in lower abundance in CRC patients. The median is represented by the solid horizontal line, the whiskers represent the minimal and maximal value, and the box represents the interquartile range.

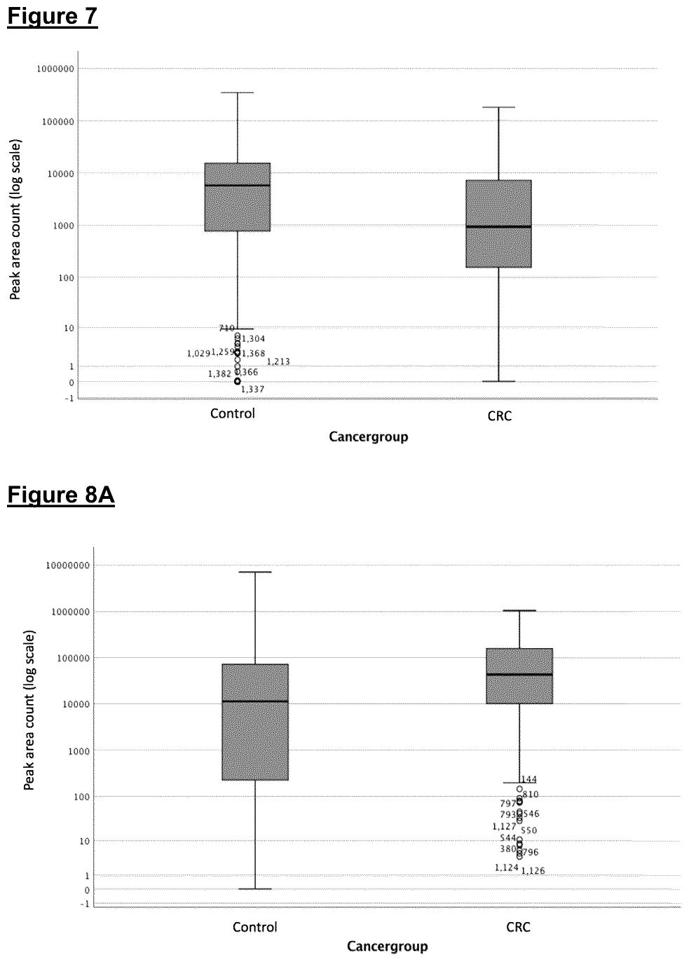

FIG. 7 shows the abundance of phenol (VOC 14), was lower in the breath of CRC patients compared to those without CRC. The median is represented by the solid horizontal line, the whiskers represent the minimal and maximal value, and the box represents the interquartile range.

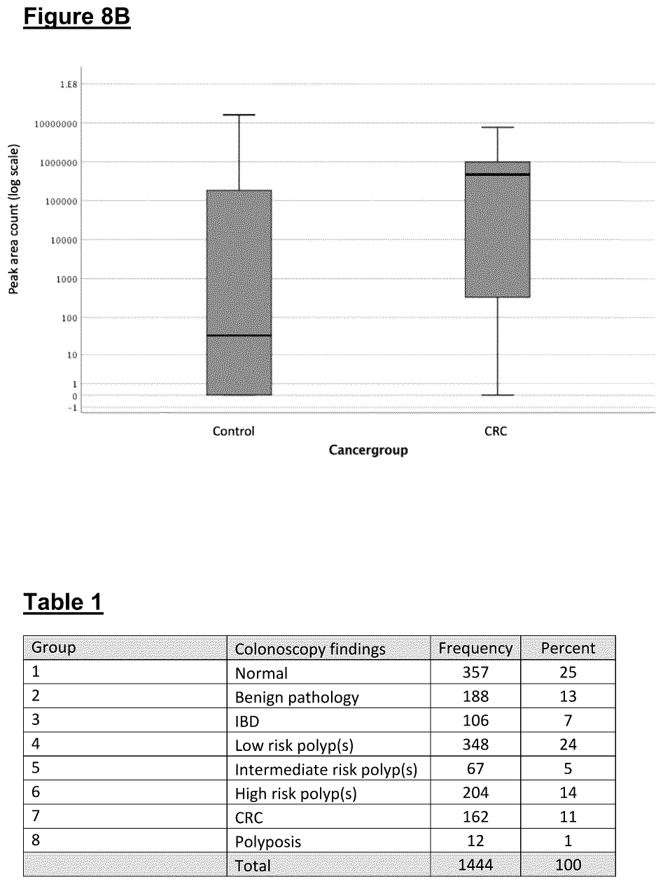

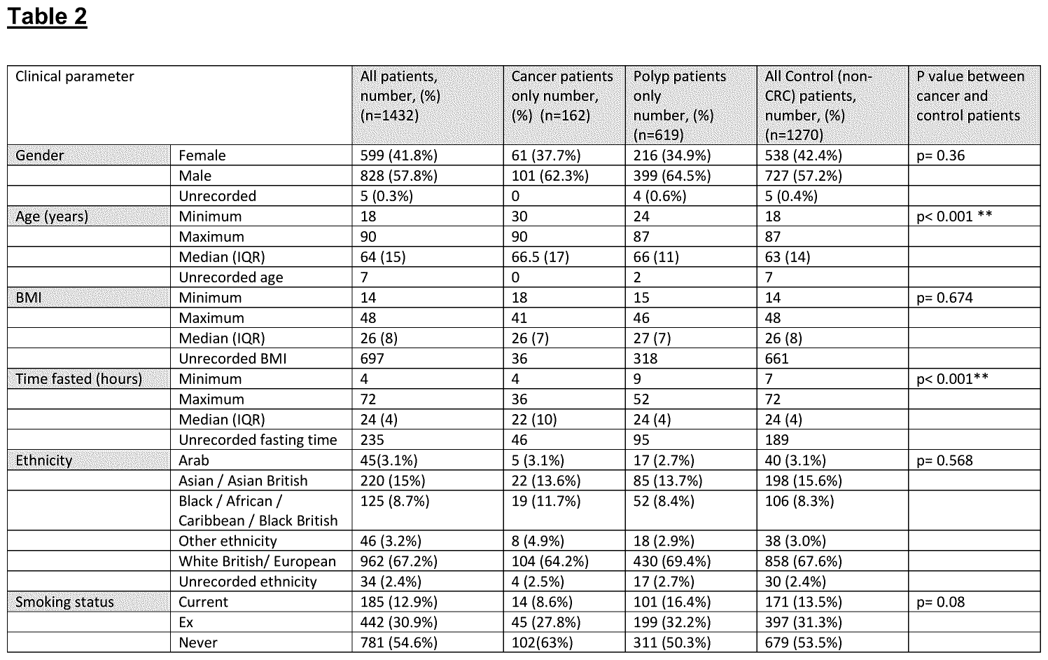

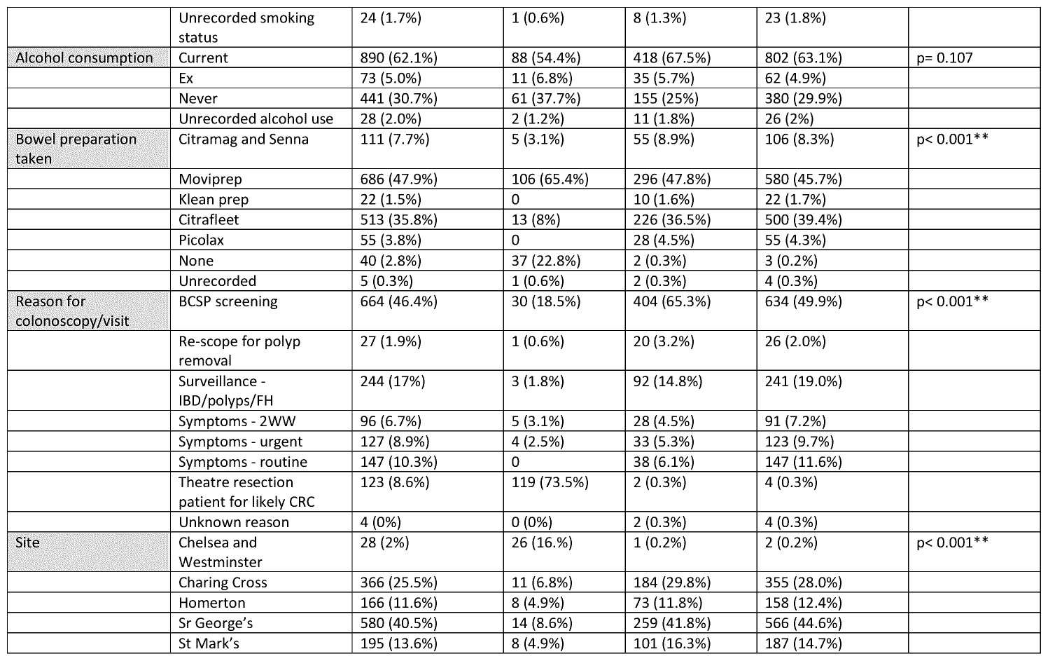

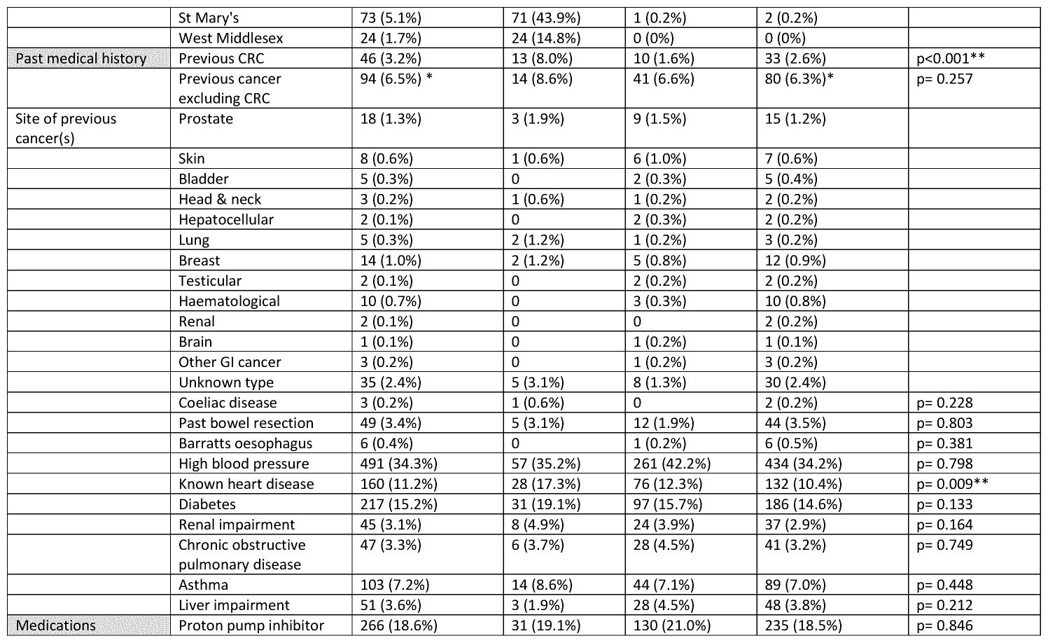

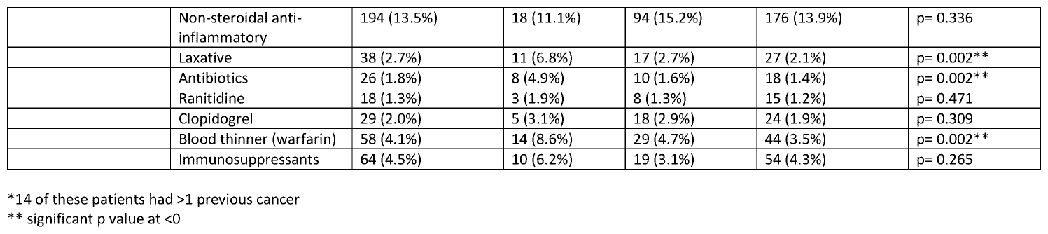

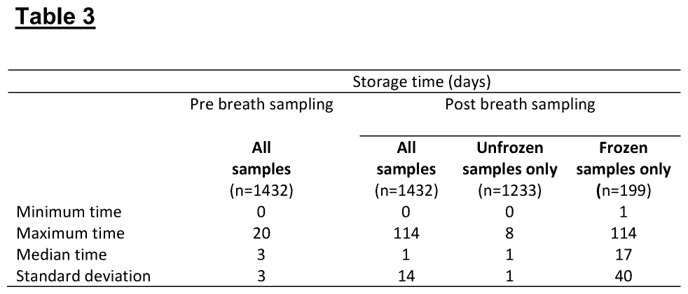

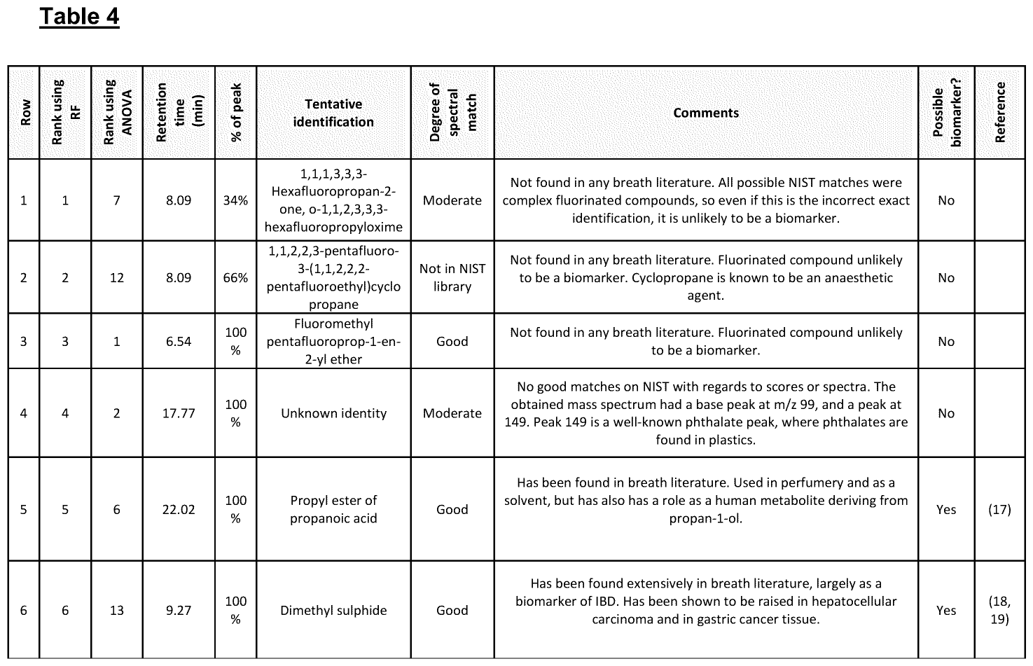

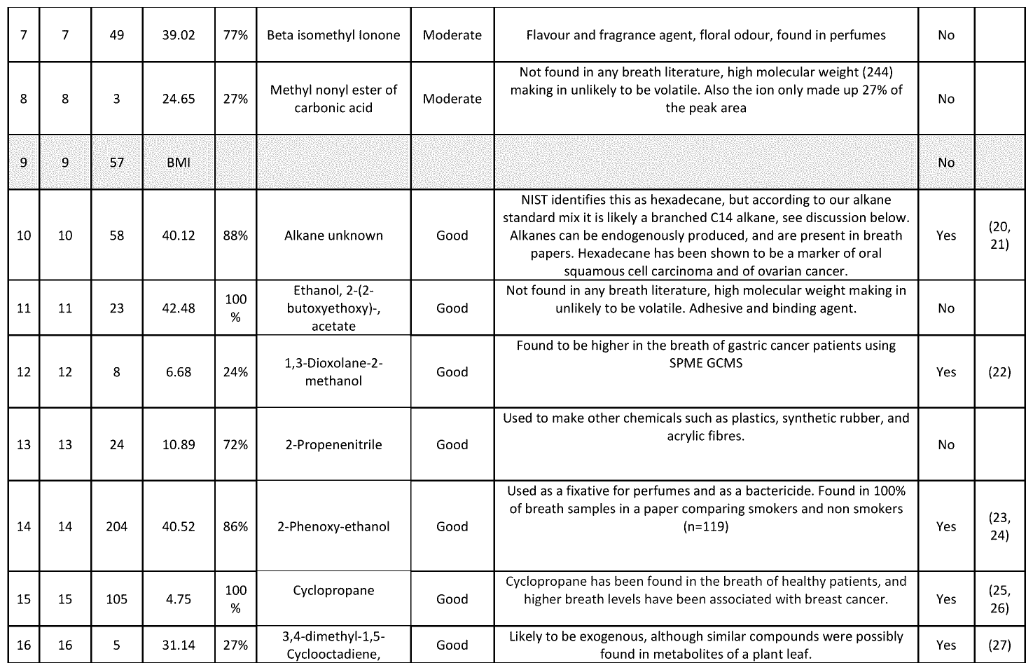

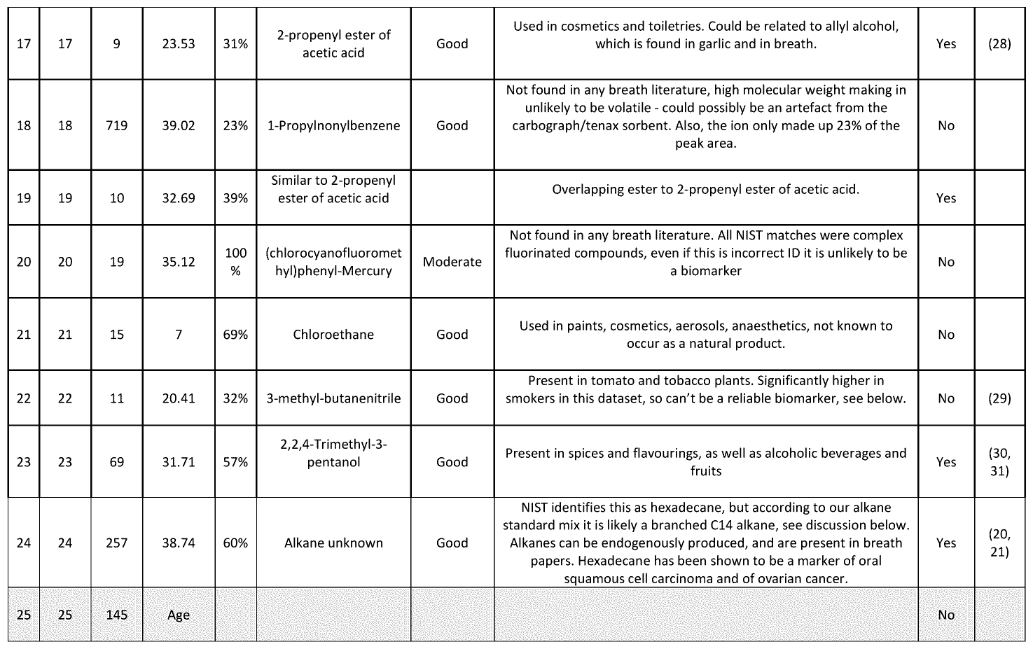

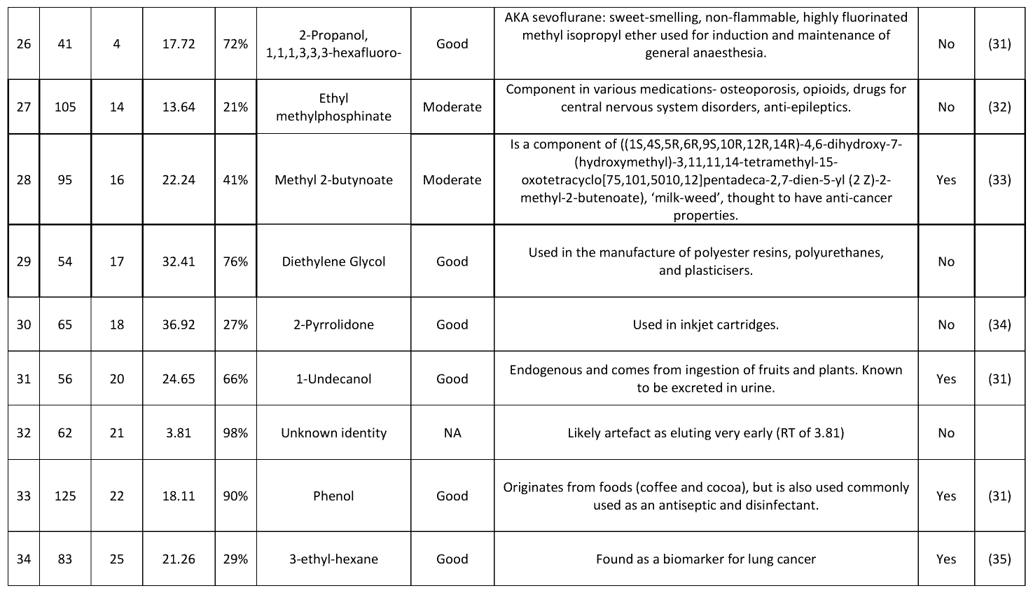

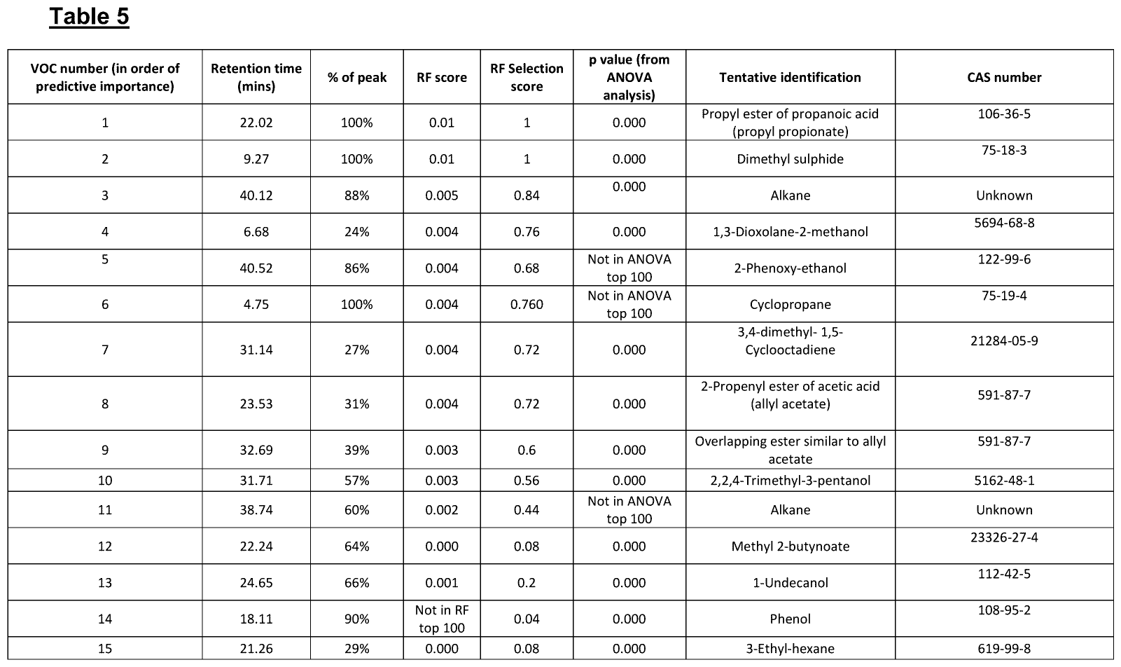

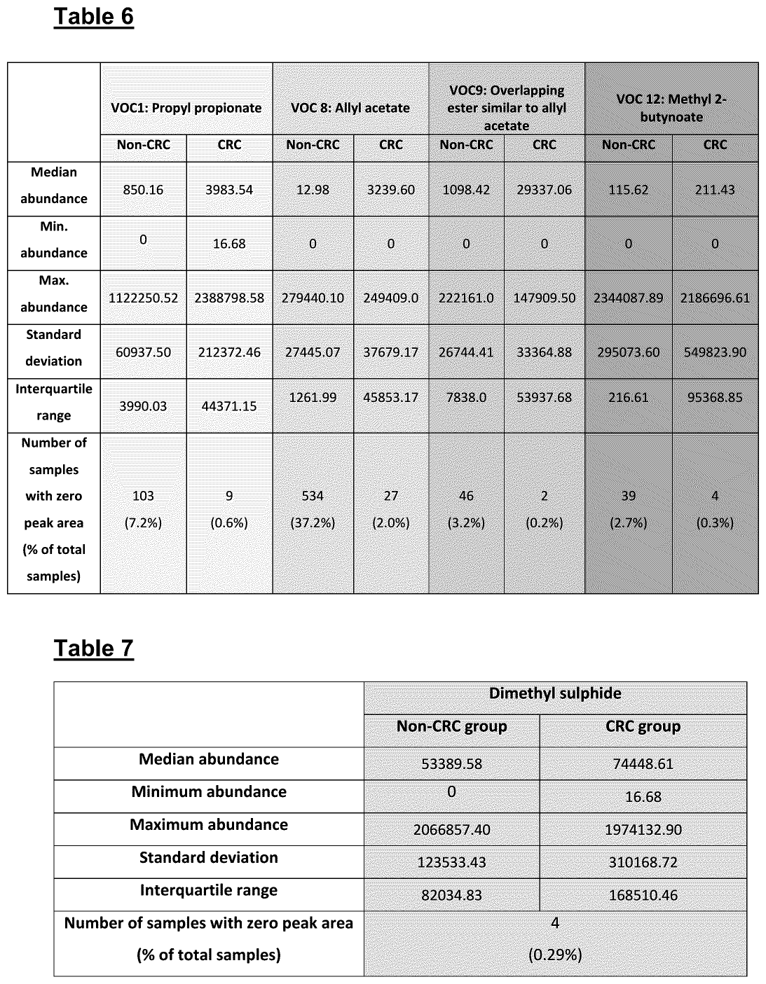

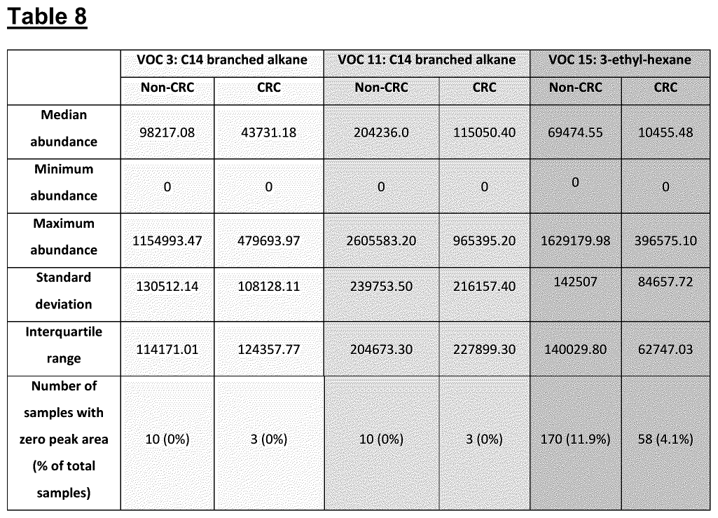

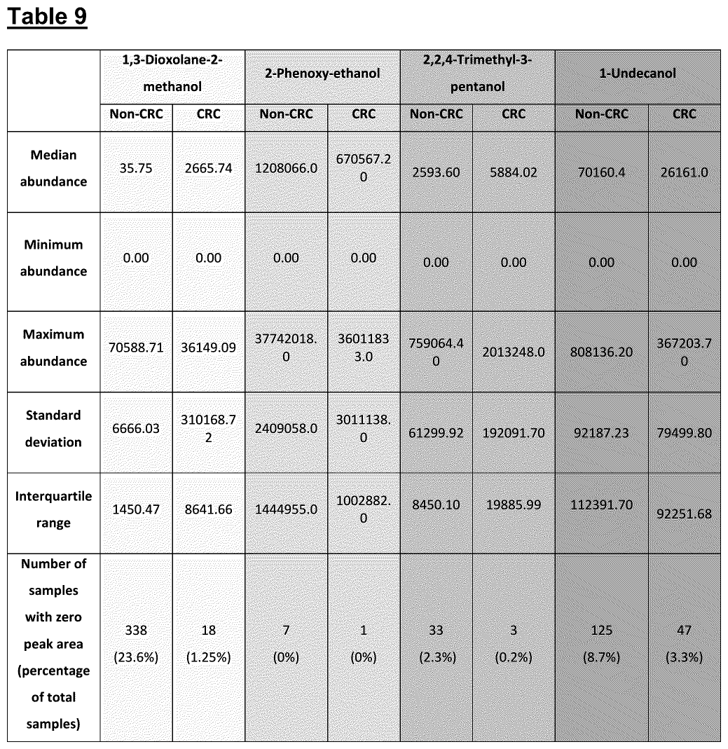

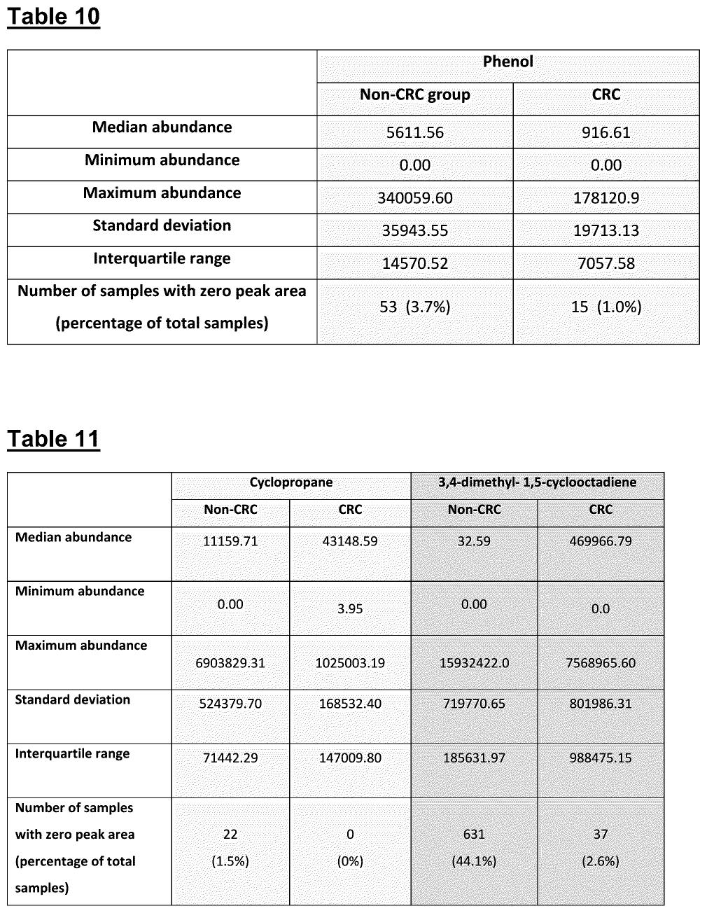

FIGS. 8A and 8B show the abundance of two non-aromatic cyclic hydrocarbons in the breath of non-CRC vs CRC patients. Both cyclopropane (VOC 6, FIG. 8A) and 3,4-dimethyl-1,5-cyclooctadiene (VOC 7, FIG. 8B), were present in significantly higher abundance in the breath of patients with CRC compared to those without CRC. The median is represented by the solid horizontal line, the whiskers represent the minimal and maximal value, and the box represents the interquartile range.

Table 1 shows the diagnosis at colonoscopy for 1444 patients.

Table 2 shows the demographics of included patients, by main pathology groups.

Table 3 shows TD tube storage time (days), n=1432.

Table 4 shows a list of top discriminating features contributing to the differentiation of CRC patients (n=162) from all positive and negative control patients (n=1270), ranked according to Random Forest (RF) and ANOVA feature selections (top 25 features from each method are listed).

Table 5 shows embodiments of the top 15 VOCs, defined as those with the potential to be CRC biomarkers, with statistical scorings.

Table 6 shows the abundance, measured in peak area count, of the four significant esters measured by TD-GC-MS between patients with (n=162) and without CRC (n=1270).

Table 7 shows the abundance, measured in peak area count, for dimethyl sulphide measured by TD-GC-MS between patients with (n=162) and without CRC (n=1270).

Table 8 shows the abundance, measured in peak area count, of three significant alkanes measured by TD-GC-MS between patients with (n=162) and without CRC (n=1270).

Table 9 shows the abundance, measured in peak area count, of four significant alcohols measured by TD-GC-MS between patients with (n=162) and without CRC (n=1270).

Table 10 shows the abundance, measured in peak area count, for phenol measured by TD-GC-MS between patients with (n=162) and without CRC (n=1270).

Table 11 shows the abundance, measured in peak area count, of two significant non-aromatic cyclic hydrocarbons measured by TD-GC-MS between patients with (n=162) and without CRC (n=1270).

### EXAMPLES

The inventors investigated the use of volatile organic compounds (VOCs) present in exhaled breath, for the prediction of colorectal cancer (CRC) and adenomatous polyps.

The objectives of this study were to: (i) collect and compare breath VOCs from a large cohort of patients with CRC, adenomatous polyps, benign diseases of the colon, and no colonic disease, as diagnosed on colonoscopy; (ii) use technologies that allow detection of VOCs at trace level; (iii) investigate the diagnostic accuracy of the breath test in a group of patients who have CRC and adenomatous polyps compared to subjects with benign diseases or normal colons, by constructing a diagnostic model; and (iv) identify and biologically characterise any significant ions.

### Materials and Methods

### Ethical Approval

The Colorectal Breath Analysis (COBRA) study was given REC approval on 28/04/17 (17EE0112), and HRA approval on 02/05/17 (East of England-Essex REC). Site Specific Assessment was also carried out at all 7 participating hospitals, with approval of the study sponsor.

In order to sample breath from patients enrolled into the English Bowel Cancer Screening Programme (BCSP), COBRA received specific approval from the BCSP Research Advisory committee (BCSP ID189, approval given on 18/01/17). In addition, COBRA was adopted into the National Institute of Health Research (NIHR) portfolio. This allowed recruitment to be conducted by NIHR affiliated research nurses.

The study was conducted in accordance with the recommendations for physicians involved in research on human subjects adopted by the 18th World Medical Assembly, Helsinki 1964 and later revisions.

### Methodology

COBRA was a prospective, non-randomised, cohort study designed to sample the breath of patients having colorectal investigations in secondary care at 7 London hospitals, over 3 years, starting on 5 Jun. 2017.

### Inclusion Criteria

Participants between the ages of 18 years and 90 years inclusive, who were able to provide informed written consent, undergoing a lower gastrointestinal endoscopy (colonoscopy) as part of their routine clinical care, or scheduled to undergo elective resection of histologically confirmed colorectal adenocarcinoma.

### Exclusion Criteria

Patients who lacked capacity or were unable to provide informed consent, and any patient below 18 years of age or over 90 years of age.

### Patient Selection—Endoscopy Unit

Patients were invited to participate in the study whilst waiting for a planned colonoscopy in the endoscopy unit of one of 4 participating London-based BCSP endoscopy centres. Patients waiting for a BCSP colonoscopy were approached preferentially, because their chances of having a colonic polyp was estimated to be around 40% [13] and chance of CRC higher than in the general population (given that all BCSP attendees were by definition faecal occult blood test-positive at the time of sampling). However, any other patients attending for a colonoscopy were also eligible, including those attending for 2 week wait (2WW) or surveillance colonoscopies. Patients sampled in the endoscopy unit were not pre-selected before the day, they were sampled as they came, and neither the study organisers nor the breath samplers had seen any of the patient's medical history or records prior to sampling them. All had been referred for a colonoscopy either on clinical grounds by their usual medical practitioner, or were attending as part of the BCSP. All patients were nil by mouth and had fasted for a minimum of 6 hours as per usual endoscopy guidelines. Patients were sampled in a side room away from other patients, in a seated position, before entering the endoscopy procedure room. This was done to avoid any effects of sedative drugs or anaesthetic throat spray present in the endoscopy room itself.

### Patient Selection—Theatres

An additional cohort of patients was approached for the study who were known to have current active CRC, specifically colorectal adenocarcinoma in situ (within the colon), with a planned upcoming surgical resection of the tumour. These patients were identified in one of 3 participating London hospitals. Included patients were not taking chemotherapy at the time of the operation. Patients were approached on the morning of their surgery to ask if they would give a breath sample before their operation. All patients were nil by mouth and had fasted for a minimum of 6 hours as per usual theatre guidelines. Patients were sampled in a side room in the surgical department away from other patients and separated from theatres, in a seated position. All breath samples were retrieved prior to the anaesthetic or surgical procedure, before transfer to the anaesthetic room.

### Breath Sample Collection

Patients were sampled in an identical fashion regardless of whether they were recruited from endoscopy or theatres. The breath test involved participants performing normal tidal breathing whilst wearing a sterile rubber facemask (single use) fitted onto the ReCIVA™ CE-marked handheld breath testing device (Owlstone, Medical Ltd, Cambridge, UK), as per the published optimised settings [14]. In brief, during exhalation, breath was entrained from the mask via four thermal desorption (TD) tubes (Markes International, Llantrisant, UK) at a flow of 200 mls/minute using inbuilt pumps (triggered by rising carbon dioxide levels), having a final volume of 500 ml per tube. The TD tubes were packed with Carbograph/Tenax sorbent phase, designed to retain VOCs. The ‘whole breath’ setting for breath fraction was chosen. After the breath test (which lasted approximately 5 minutes), the TD tubes were sealed by screwing brass caps onto each end with a specific spanner, to ensure that the breath VOCs were trapped onto the sorbent in the TD tube and could not desorb and escape. Researchers also filled out a clinical details form, detailing past medical history, body mass index (BMI), medications and key information such as smoking status and last meal. Sets of four capped TD tubes were then placed in plastic sealed sampling bags, labelled with the unique study identifier, and the date, time and site of sampling.

### Specimen Analysis

Breath VOCs were analysed using two mass spectrometry techniques: Proton-Transfer-Reaction Mass Spectrometry (PTR-MS) and Gas Chromatography Mass Spectrometry (GC-MS). Three of the four TD tubes from each patient were analysed using PTR-MS (using three different reagent ions H₃O⁺, NO⁺, O₂⁺), and one TD tube using the GC-MS. The GC-MS Agilent 7890B GC with 5977A MSD (Agilent Technologies, Cheshire, UK) was used, coupled with a Markes TD-100 (Markes Ltd, Llantrisant UK) TD unit. GC-MS analysis was performed with a two-stage desorption method using a constant flow of helium at 50 ml/min and a cold trap system (U-T12ME-2S, Markes International Ltd, Llantrisant, UK). Samples were then transferred to the GC system by a capillary heated at 200° C. The chromatographic column employed for compound separation was a Zebron ZB-642 capillary column (60 m×0.25 mm ID×1.40 μm df; Phenomenex Inc, Torrance, USA).

GC-MS data were extracted using MassHunter software version B.07 SP1 (Agilent Technologies) and further analysis was conducted using a custom designed in-house built software MSHub [15, 16]. VOC peak identification was performed using the NIST mass spectral library (National Institute of Standards and Technology version 2.0) [17].

GC-MS is considered the gold standard for the analysis of VOCs in breath. For this reason, the inventors chose to use this platform, characterised by high reliability and good VOC identification performance. PTR-MS is a novel technique, used in environmental research. PTR-MS is characterised by high-throughput and real-time results. In contrast to GC-MS, PTR-MS provides direct quantification of compounds, without the need for external calibration. These aspects make the use of the two techniques complementary. GC-MS offers reliable compound identification while PTR-MS offers high-throughput analysis and quantitative results. For this reason, GC-MS was used as a “discovery” technique, while PTR-MS was used to provide a fast real-time method. For the biomarker identification purposes, only GC-MS data will be discussed. The ReCIVA® breath sampler has the ability to collect four breath samples simultaneously, allowing two mass spectrometry platforms to be used without adding additional breath sampling time for the patients.

### Data Analysis

### Demographics and Clinical Data

Potential confounding factors across the CRC and control groups were evaluated using the Mann-Witney U test for continuous variables and X test for discrete variables. P<0.05 was used to assign statistical significance. This statistical analysis was performed using the statistical software SPSS (version 25, IBM).

### Breath VOC Data

The raw data from the TD-GC-MS analysis were processed with MSHub, a custom-made spectrum processing program, made at Imperial College London [15, 16]. This was a dataset-based spectral deconvolution tool for use within the Global Natural Product Social Molecular Networking (GNPS) environment. The steps by which MSHub processed the raw data were: intra/inter-sample mass drift correction, noise filtering and baseline correction, inter-sample peak alignment, peak detection and integration, NMF deconvolution then peak deconvolution [15, 16]. This gave an output that consisted of multiple ions (or VOCs) labelled as numbered features, their retention times, and the peak area count of each feature in each patient's breath sample. Not all features were present in all samples. In addition, there were some features identified which were ions that made up a very small proportion of the total peak for a given retention time, present in the minority of samples. Ions that made up less than 20% of the total peak were included in the statistical analysis, but were not considered in the list of top differentiating features in the comparisons of different clinical patient groups. The target ions from the obtained spectra were matched using the on-line NIST library for potential identification [17]. The MSHub utilises a one-layer neural network for GC deconvolution, which allows information to be extracted across the entire dataset (as opposed to a single spectrum at a time) and thus utilises all of the spectral information within the data, a strategy that is particularly successful for large-scale studies.

### Statistical Analysis

Both univariate and multivariate data analysis techniques were applied to the results to (i) identify VOC components with the best discriminating ability between the groups; and (ii) to develop a multivariate discriminant analysis model.

The Mann-Whitney U test was used to compare the measured VOC levels between selected groups, namely CRC vs non-CRC groups, or to investigate potential confounding factors such as sampling environment or anatomical site of tumours. A p value <0.05 was taken as the level to indicate statistical significance.

A non-parametric (Kruskal-Wallis) ANOVA test was used to compare the measured VOC levels (VOCs represented as ions) between all 7 of the included study pathology groups (grouped according to diagnosis as per colonoscopy result). This was done to determine if any of the 7 patient groups contained an abundance of a VOC that was statistically significant in differentiating between groups. A p value <0.05 was taken as the level to indicate statistical significance. This basic statistical analysis was performed using the statistical software SPSS (version 25, IBM) [18].

Clinical parameters that required further investigation to establish any confounding influences on VOC abundance, such as fasting time and T stage of the tumour, were investigated with Pearson's correlation coefficient (in the case of fasting time), and by plotting VOC abundance trends in the case of tumour T-stage comparisons. This was done using SPSS (version 25, IBM) and Microsoft Excel v16.43.

### Machine Learning Prediction Models

A high performance computer facility at Imperial College London was utilised to run a machine learning pipeline to process all of the abundance data of unidentified features so in each patient's breath sample (1024 features were identified in each sample), and the extensive metadata for each patient. The data was normalised, variance stabilised and log-transformed as part of the machine learning pipeline. Random forest, alphanet, SVM, lasso and elastic machine learning prediction methods were used independently to compare every combination and permutation of pathology group. The same analyses were repeated also for patients of age 40-59 years, 45-65 years, 50-69 years and 70-89 years, as well as all ages together, to investigate whether age was confounding the VOC data. The prediction models took into account a wide range of clinical variables between groups. These included patient factors: age, number of hours of fasting, BMI, ethnic origin, gender, smoking status, weekly alcohol consumption, type of bowel preparation taken before colonoscopy/surgical resection and family history of CRC. Sampling related factors were also included: the method by which TD tubes had been cleaned before sampling (using the standard TC20 conditioning unit, or using the PTR-MS instrument itself), the storage time of the TD tube from conditioning to breath sampling, the storage time post-sampling until MS analysis, and the number of days the TD tube was stored in the freezer (if applicable). Factors that were directly linked to outcome were excluded from the prediction model, such as reason for colonoscopy, sampling site, and any data linked to colonoscopy findings. Details of past medical history and medications were not input into the model, as answers were too heterogeneous.

Receiver operating characteristic (ROC) curves were used to determine the accuracy of a diagnostic test in classifying those with and without colorectal disease. The ROC curves were generated based on 25 runs: 5 repeats of 5-fold stratified K-fold splits with re-shuffling between splits. This meant that samples were shuffled and then split into 5 groups. Each group was then used in turn as a test set, while the other 4 were the training set. Feature selection and model building (machine learning) were performed on a training set each time (80% of the data) and then applied to the test set (20% of the data) to produce the statistics. This was repeated 5 times and then the results from different runs were averaged to get ROC curves and error estimates. Because this analysis method was chosen, each time the data was split, the selection of significant features varied slightly.

The average number of times any given feature was selected as a predictive/significant feature was displayed as a feature selection score. If a feature was independently selected to be a differentiating feature regardless of how the data was split, the selection so score would be higher. A higher score therefore meant that the feature in question was more likely to be a true feature differentiating marker for CRC and non-CRC, as opposed to a chance finding.

In addition, in the case of Random Forest (RF) method, the contribution that each feature made to the prediction model was represented by the RF score. The scores for all features contributing to the generation of the predictive model always added up to 1 (by definition). The highest scoring features therefore represented the most important in terms of differentiating the comparator groups. The score was calculated by computing the normalised total reduction of the criterion brought about by that feature (also known as the Gini importance) [19].

### Results

### Patient Group Allocations

Patients were grouped according to the findings on the colonoscopy that they had on the day of attendance. The benign pathology group had minor non-inflammatory findings; haemorrhoids, benign non inflammatory anal fissures, diverticular disease or benign diverticular strictures. The inflammatory bowel disease (IBD) group consisted of ulcerative colitis (UC), Crohn's disease, unspecified colitis or infective colitis, of any severity. Some patients had a history of IBD in their records, but had a normal colonoscopy with normal biopsies. These patients were allocated to the normal group. Polyps were stratified into high, intermediate and low risk of development into CRC using adapted criteria taken from the British Society of Gastroenterology polyp surveillance guidelines 2002 and the more recent guidance on sessile serrated polyps from 2017 [20, 21].

Low risk polyp patients were those with 1-2, small (<1 cm) tubular adenomas with low grade dysplasia, or sessile serrated polyps (SSPs)<1 cm with no dysplasia. Intermediate risk polyp patients were those with 3-4 small tubular adenomas, with low grade dysplasia, or at least one adenoma>1 cm, with low grade dysplasia, or SSPs >1 cm, with no dysplasia. High risk polyp patients were those with >5 adenomas, or >3 adenomas where at least one is >1 cm, or any adenoma with high grade dysplasia, or any adenoma with any villous change (including tubulovillous adenomas), or any SSP with evidence of dysplasia.

CRC patients all had colorectal adenocarcinomas, where size, site, grade of tumour and TNM stage were documented. Polyposis patients were those with an existing diagnosis of polyposis (familial adenomatous polyposis (FAP) where colectomy had been refused, serrated polyposis, Lynch syndrome, juvenile polyposis or MUYTH associated polyposis). This was a heterogeneous group of patients as whilst some had >100 polyps present on the colonoscopy that day, others had only one 1 or 2 polyps, largely due to very frequent surveillance and polypectomy, and a significant number had had resections of a part of the colon already. Some were likely to have had upper gastrointestinal polyps also. Because of the variation in colonoscopy findings within this group, and the difficulty of confidently excluding a CRC in those with many polyps, the polyposis group was excluded from the statistical analysis.

### Colonoscopy Findings

1444 patients had breath samples analysed by GC-MS (see Table 1 for their diagnoses). 162 had CRC (11%), and 631 (43.7%) had polyps. As explained above, the polyposis group was small and very heterogeneous, and therefore, was excluded from subsequent analyses. 1432 patients were therefore included in the statistical analyses (unless stated otherwise).

Colonoscopic diagnosis was determined as per the most significant finding. The diagnostic group hierarchy was CRC, polyposis, high risk polyp(s), intermediate risk polyp(s), low risk polyp(s), IBD, benign pathology, normal. This meant that if a patient had IBD and a polyp, regardless of whether it was active IBD or not, they were placed in the appropriate polyp group. In the same way, a high risk polyp categorised patient could also have a diverticulum or haemorrhoid. It is known that active IBD can alter the VOCs in breath [22] so this could represent a confounder, however, there was in reality very little cross over between polyps and IBD, affecting 13 patients only.

### Demographics

The control group (n=1270) included all positive and negative controls combined for the purposes of statistical comparison against the CRC group (n=162). 57.8% of the recruits in this study were male, with no significant difference in gender distribution between the CRC and control groups. CRC patients were significantly older than control patients, at 66.5 years in comparison to 63 years respectively (p<0.001). The majority of the patients were white British or European origin, and most were non-smokers, were current consumers of alcohol, with a median BMI of 26. There was no statistically so significant difference between the distribution of these variables between the CRC and control groups. Although the median fasting time for CRC and control groups was similar, there was a statistically significant difference between groups, where the CRC group fasted for less time (p<0.001). The majority of patients had Moviprep as bowel preparation before their colonoscopy or theatre procedure. There was a significant difference in bowel preparation distribution between cancer and control groups (p<0.001), largely because a narrower range of bowel preparations was used for pre-theatre patients and because 37 CRC patients had no bowel preparation before the breath test. The ‘reason for colonoscopy/visit’ and ‘site sampled at’ results were statistically significant between CRC and control groups because a significant proportion of CRC patients came from recruitment from theatres.

In the endoscopy unit, the study targeted BCSP patients primarily; 30 CRCs were detected at BCSP colonoscopies, giving a 4.5% CRC pick up rate from BCSP patients, lower than in the literature [13]. This was slightly less than the CRC pick-up rate in the 2WW patient group (5 out of 96 colonoscopies=5.2%). Other CRCs were detected in the surveillance (n=3), urgent symptoms (but not 2WW) (n=4) and re-scope for polyp removal groups (n=1), and none in the routine symptoms group. The rest of the cancer cases (n=119) were sampled pre-theatre having been identified for the study beforehand, representing an enriched cohort. As expected, the highest yield of polyp patients came from the BCSP and polyp surveillance groups. The polyp pick-up rate was 63% in the BCSP patients, higher than in the literature (this calculation included 17 of the BCSP-diagnosed CRC patients, who also had polyps found at colonoscopy) [13]. Past medical history and medication use were also recorded. There was a statistically significant difference in the number of patients who had had CRC in the past, in the CRC group. These 13 patients therefore represented CRC luminal recurrence (in addition to extra-intestinal recurrence in some cases). Other statistically significantly increased co-morbid factors for the CRC group were the prevalence of known heart disease, laxative use, recent antibiotic use and warfarin (or other anticoagulant) use. Other comorbidities and medications used were comparable between CRC and control groups, see Table 2.

### Clinical Details of Colorectal Cancer Patients

Cancer specific details were recorded for all CRC patients. Most CRCs were left sided (62%), and over half (64%) were late stage cancers T3 and T4, mostly with an N score of 0 to 1 and mostly without metastases. The range of size of tumour was 6 mm to 130 mm, median size 38.5 mm (at greatest tumour diameter). 80% were moderately differentiated adenocarcinomas.

The route of diagnosis of the CRC influenced what stage the CRC was. CRCs picked up in the BCSP were quite evenly distributed in terms of T stage, but the proportion of early cancers was higher in this group than any other (48% of BCSP cancers were T stage 1 or 2). This contrasted to the CRC patients who had been recruited via the theatre route. These patients tended to be symptomatic and a very high proportion of them (72%) had T stage 3 or 4 cancers. This was an expected finding given that the BCSP is aimed at performing colonoscopy in asymptomatic individuals. In patients diagnosed with CRC, their age did not seem to necessarily correlate with the T stage that they were diagnosed at.

### Sample Processing Times

The storage time of the cleaned TD tube prior to sampling, and the storage time of the breath sample on the TD tube before analysis by GC-MS are detailed in Table 3. There was no significant difference on a Kruskal-Wallis comparison (IBM, SPSS statistics version 25) between the 7 pathology groups with regards to storage of the TD tube prior to sampling (p=0.84), or post sampling (p=0.93), for all samples. No TD tubes were frozen prior to sampling, but post sampling 199 TD tubes were frozen for 1 to 114 days (median 17 days, standard deviation 40 days) before analysis, due to instrument down time/unavailability. For frozen tubes there was also no significant difference in post-sampling storage time between CRC and control groups (p=0.23). All tubes used for a patient sample (4 tubes per patient) were always conditioned/cleaned at the same time.

### Results of Initial Univariate Statistics

1024 features (VOCs) were identified in breath, and their peak area counts were tabulated by the MSHub programme [15, 16].

To start, a Kruskal-Wallis test was done performed to determine if any of the 7 patient groups contained an abundance of a VOC that was statistically significant in differentiating between groups. 291 ions were found to be differentiating, with a p<0.05.

A Mann Witney U analysis was performed for CRC (n=162) vs control (n=1270) patients. 336 features (ions) were found to be differentiating, with a p<0.05. 95% of the features detected as discriminatory by the Kruskal-Wallis test were overlapping with the features found by the Mann Witney U analysis, suggesting that it was the cancer group accounting for the significant differences in most cases. Groups were therefore interrogated in depth using advanced machine learning prediction models, where the clinical metadata was also incorporated as variables.

### Results of Machine Learning Prediction Model—CRC Vs Non-CRC

The first machine learning analysis that was performed compared all detected VOCs from the CRC patients (n=162) against all those from the non-CRC (control) patients (n=1270), using GC-MS data. The strongest model for the prediction of CRC vs non-CRC was the machine learning elastic method, which could predict the CRC patients with a sensitivity of 0.77 (+−0.02), a specificity of 0.87 (+−0.01), a negative predictive value of 0.97 (+−0.00) and an accuracy of 0.86 (+−0.01). The area under the receiver operating curve (ROC) was 0.87 (+−0.01); see FIG. 1.

The non-CRC patient group comprised of both positive and negative controls. The negative controls (with normal colons at endoscopy) numbered 357. Positive controls with benign disease, IBD, or low/intermediate/high risk polyps at endoscopy numbered 913.

This ROC curve in FIG. 1 was calculated based upon the results of a cross-validation method of 5 cycles of 5-fold stratified k-fold splits with reshuffling. This means that the ROC curve was the average (mean) of ROC curves from individual runs, each with a slightly different feature selection and machine learning model. The area under the curve (AUC) for each individual cycle is shown in the key. Up to 99 features were used to generate this ROC curve, as determined by the machine learning algorithm, where “features” referred to individual ions but also individual clinical variables, i.e. any component that contributed to the separation of the groups. The number of features used was demonstrated by the RF selection score (the average number of times any given feature was selected for the method), where a score of 1 would mean that the feature in question was selected 100% of the time.

The top 25 chemical features, as well as 2 clinical features that achieved the highest discriminatory scorings for CRC vs non-CRC are listed in Table 4. These were the features giving the highest contribution to the creation of the ROC curve (FIG. 1). Features were ranked using both RF selection and ANOVA, hence why the list of top 25 ions was slightly different depending on which method was chosen. This was expected because ANOVA dealt with each feature one at a time and did not take feature cross-correlations or any other information into account, whereas RF constructed a model based on the entire ensemble of features and thus could take feature interactions into account. Both lists' features were interrogated. Features were identified by comparing the obtained mass spectra to possible matches suggested by the NIST database [17]. If the two mass spectra showed the same distribution and intensity of ions, then this was a match and the compound could be identified with a good degree of confidence. Where there was an imperfect but close spectral match, compound identification was tentative. During the GC-MS analysis deconvolution, at any given retention time, a peak could be split into two (or more) peaks with different fragmentation patterns. The “percentage of peak” column in Table 4 shows how much of the original peak was explained by this new deconvolved peak. The lower the percentage, the less contribution this peak had and the less resolved it was. When the value was 100% there was a single peak, completely resolved. Any peaks that contributed to less than 20% of the original peak were excluded.

From the comprised list (Table 4) of 25 top cancer-differentiating ions from two different machine learning prediction models (ANOVA and RF), a short list was created. These short-listed ions were manually selected with the following criteria: (i) they could be considered endogenous (ii) they had a physiological role that could explain their involvement in CRC. 3-methyl-butanenitrile was not considered as a compound of potential importance because of its presence in tobacco plants [23], leading to interrogation of the COBRA dataset; there was a significantly higher abundance of this compound in smokers (n=185) compared to non-smokers (n=781), p=000009 using a Mann Witney U test. The identification of this compound using the NIST library showed a good degree of confidence, since we obtained a good spectral overlap. 3-methyl-butanenitrile was therefore excluded as a potential CRC marker. Table 5 details the 15 ions that were taken forward as potential VOC biomarkers for further investigation, with their statistical scorings. Applying just these top 15 features in isolation on the dataset, a ROC curve with an AUC of 0.83, and a 95% confidence interval of 0.79-0.86 was obtained, see FIG. 2.

### CRC Vs No Colorectal Pathology Analysis

The same machine learning analysis as performed above was repeated for the CRC group (n=162) vs the normal/benign colorectal pathology group only (n=545). This group had either normal colonoscopies or benign findings such as a haemorrhoid, diverticular disease or a benign non-IBD associated anal fissure. Interestingly, 23 of the resultant top 25 features using RF selection overlapped with the top 25 features for the larger CRC vs non-CRC comparison described above, suggesting that the markers found could be truly CRC-specific and unaffected by other colorectal pathologies such as IBD and polyps. The two new VOCs that were not in the pre-existing list were pentafluoroethane, similar to other fluorinated compounds found in the CRC vs non-CRC comparison, see Table 4, and 2-methyl-2-propanol. Each of the top 15 discriminating ions for CRC was explored in detail, within their chemical groups.

### The Esters

VOCs 1, 8, 9 and 12 were tentatively identified as propyl propionate, allyl acetate, a similar overlapping ester to allyl acetate, and methyl 2-butynoate. All four of the obtained esters were present in significantly higher abundance in the breath of patients with CRC (n=162) compared to those without CRC (n=1270). The ion peak area counts are given for both groups in Table 6, and representative boxplots of the distributions in each group are demonstrated in FIGS. 3A-3D.

### Sulphur Compounds

VOC2 was identified as dimethyl sulphide, with a good match between the obtained mass spectrum and the NIST database. It has a chemical formula of C₂H₆S₂ and an m/z of 63. Dimethyl sulphide was found to be present in a significantly higher abundance in the breath of patients with CRC (n=162) compared to those without CRC (n=1270). The obtained peak area count for the two study groups are given for both groups in Table 7, and representative boxplots of the distributions in each group are demonstrated in FIG. 4. The boxplot shows that the abundance of dimethyl sulphide was higher in CRC patients, but that there was some overlap.

### The Alkanes

VOCs 3, 11 and 15 were identified as two unidentified alkanes and 3-ethyl-hexane respectively. All three of these alkanes were significantly lower in CRC patients than in non-CRC patients. However, alkanes are notoriously difficult to identify as the mass spectra are very similar using GC-MS (as demonstrated by the mass spectra of VOC 3 and 11), so the spectra alone are not enough to be able to give unequivocal identification. To aid with this, a standard mix of 12 straight chain alkanes, from C8 to C20 (octane, nonane, decane etc.) was analysed by GC-MS to obtain specific retention times, for identification purposes. Retention time is dependent upon volatility and affinity for the column, where more volatile compounds will have a lower retention time. The retention times for the alkane standards were, as expected, aligning in sequence as molecules became less volatile. The retention time peaks for the two unidentified alkanes discovered in the COBRA study fell between the retention time peaks for C13 and C14 alkanes. This makes them very likely to be C14 alkanes, but with a branched carbon chain, causing them to elute from the column slightly earlier than the C14 unbranched alkane, as they are slightly less retentive due to their stereochemistry. The conclusion was therefore that both VOCs are likely to be branched chain alkanes of C14.

All three alkanes were found to be present in significantly lower abundance in the breath of patients with CRC (n=162) compared to those without CRC (n=1270). The obtained peak area count for the two study groups are shown in Table 8 and representative boxplots of the distributions in each group are demonstrated in FIGS. 5A-5C.

### The Alcohols

VOCs 4, 5, 10 and 13 were identified as 1,3-Dioxolane-2-methanol, 2-Phenoxy-ethanol, 2,2,4-Trimethyl-3-pentanol and 1-Undecanol respectively. These are all alcohols, and all had good matches with corresponding NIST library mass spectra, particularly in the case of VOC 4, 5 and 14, making their tentative identities more confident.

VOCs 4 and 10 were found to be present in significantly higher abundance in the breath of patients with CRC (n=162) compared to those without CRC (n=1270). VOCs 5 and 13 were found to be in lower abundance in CRC. The obtained peak area count for the two study groups are given in Table 9, and representative boxplots of the distributions in each group are demonstrated in FIGS. 6A-6D.

### Phenol

VOC 14 was identified as phenol. Phenol was found to be in lower abundance in CRC patients compared to controls. The obtained peak area count for the two study groups are given in Table 10, and the representative boxplot of the distributions in each group are demonstrated in FIG. 7.

### The Non-Aromatic Cyclic Hydrocarbon

VOCs 6 and 7 were identified as cyclopropane and 3,4-dimethyl-1,5-cyclooctadiene. Both cyclopropane and 3,4-dimethyl-1,5-cyclooctadiene were found to be present in significantly higher abundance in the breath of patients with CRC (n=162) compared to those without CRC (n=1270). The obtained peak area count for the two study groups are given in Table 11 and representative boxplots of the distributions in each group are demonstrated in FIGS. 8A and 8B.

### Conclusions

The findings support a clear association between a number of VOCs in the breath and the presence of colorectal cancer. In particular, the results demonstrate that exhaled breath could be used to detect the presence of CRC of all stages from positive and negative controls with an area under the ROC curve of 0.87, a sensitivity of 77%, a specificity of 87% and a negative predictive value of 97%, in 1432 patients attending hospital for a colonoscopy or for CRC resection in theatre.

The 15 VOCs identified as significant CRC biomarkers in Table 5 included dimethyl sulphide, phenol, and compounds from the ester, alcohol, alkane and non-aromatic cyclic hydrocarbon chemical classes. These 15 VOCs together were able to predict the presence of CRC from positive and negative controls using breath with an area under the ROC curve of 0.83. Accordingly, the results show promising potential of breath VOC testing as a diagnostic tool for colorectal cancer and provide the basis for a larger multicentre trial, moving a step closer to the implementation of this innovative and highly acceptable tool for reliable and non-invasive CRC and polyp detection into clinical practice.

### REFERENCES

1. Torre L A, Bray F, Siegel R L, et al. Global cancer statistics, 2012. CA Cancer J Clin 2015; 65: 87-108.
2. Ewing M, Naredi P, Zhang C, et al. Identification of patients with non-metastatic colorectal cancer in primary care: a case-control study. Br J Gen Pract 2016; 66: e880-e886.
3. Lieberman D A, Weiss D; Veterans Affairs Cooperative Study Group 380. One-time screening for colorectal cancer with combined fecal occult-blood testing and examination of the distal colon. N Engl J Med 2001; 345: 555-560.
4. Imperiale T F, Ranshoff D F, Itzkowitz S H, et al. Fecal DNA versus fecal occult blood for colorectal-cancer screening in an average-risk population. N Engl J Med 2004; 351: 2704-2714.
5. Allison J E, Tekawa I S, Ransom L J, et al. A comparison of fecal occult-blood tests for colorectal-cancer screening. N Engl J Med 1996; 334: 155-159.
6. Allison J E, Sakoda L C, Levin T R, et al. Screening for colorectal neoplasms with new fecal occult blood tests: update on performance characteristics. J Natl Cancer Inst. 2007; 99: 1462-1470.
7. Imperiale T F, Ransohoff D F, Itzkowitz S H, et al. Multitarget stool DNA testing for colorectal-cancer screening. N Engl J Med 2014; 370: 1287-1297.
8. Nakhleh M K, Amal H, Jeries R, et al. Diagnosis and classification of 17 diseases from 1404 subjects via pattern analysis of exhaled molecules. ACS Nano 2017; 11: 112-125.
9. Kumar S, Huang J, Abbassi-Ghadi N, et al. Mass spectrometric analysis of exhaled breath for the identification of volatile organic compound biomarkers in esophageal and gastric adenocarcinoma. Ann Surg 2015; 262; 981-990.
10. Altomare D F, Di Lena M, Porcelli F, et al. Exhaled volatile organic compounds identify patients with colorectal cancer. Br J Surg 2013; 100: 144-150
11. Spanel P, Smith D. Selected ion flow tube mass spectrometry for on-line trace gas analysis in biology and medicine. Eur J Mass Spectrom 2007; 13: 77-82
12. Spanel P, Smith D, Progress in SIFT-MS: breath analysis and other applications. Mass Spectrom Rev 2011; 30: 236-267
13. Logan R F, Patnick J, Nickerson C, Coleman L, Rutter M D, von Wagner C, et al. Outcomes of the Bowel Cancer Screening Programme (BCSP) in England after the first 1 million tests. Gut. 2012; 61(10):1439-46.
14. Doran S L F, Romano A, Hanna G B. Optimisation of sampling parameters for standardised exhaled breath sampling. J Breath Res. 2017; 12(1):016007.
15. Aksenov A A, Laponogov I, Zhang Z, Doran S L F, Belluomo I, Veselkov D, et al. Algorithmic Learning for Auto-deconvolution of GC-MS Data to Enable Molecular Networking within GNPS. bioRxiv. 2020:2020.01.13.905091.
16. Aksenov A A, Laponogov I, Zhang Z, Doran S L F, Belluomo I, Veselkov D, et al. Auto-deconvolution and molecular networking of gas chromatography-mass spectrometry data. Nature Biotechnology. 2020.
17. Shen V K, Siderius, D. W., Krekelberg, W. P., and Hatch, H. W. NIST Standard Reference Simulation Website. Gaithersburg M D: National Institute of Standards and Technology
18. Corp I. IBM SPSS Statistics for Mac. 25.0 ed. Armonk, NY: IBM Corp.; 2017.
19. Pedregosa F, Varoquaux G, Gramfort A, Michel V, Thirion B, Grisel 0, et al. Scikit-learn: Machine Learning in Python. J Mach Learn Res. 2011; 12(null):2825-30
20. Atkin W S, Saunders B P. Surveillance guidelines after removal of colorectal adenomatous polyps.

Gut. 2002; 51(suppl 5):v6-v9.
21. East J E, Atkin W S, Bateman A C, Clark S K, Dolwani S, Ket S N, et al. British Society of Gastroenterology position statement on serrated polyps in the colon and rectum. Gut. 2017; 66(7):1181-96.
22. Hicks L C, Huang J, Kumar S, Powles S T, Orchard T R, Hanna G B, et al. Analysis of Exhaled Breath Volatile Organic Compounds in Inflammatory Bowel Disease: A Pilot Study. Journal of Crohn's and Colitis. 2015; 9(9):731-7.
23. Leffingwell J C A E. Volatile constituents of Perique tobacco. Journal of Environmental, Agricultural and Food Chemistry. 2005; 4(2):899-915.

1. A method for diagnosing a subject suffering from colorectal cancer, or a pre-disposition thereto, or for providing a prognosis of the subject's condition, the method comprising analysing the concentration of a signature compound in a bodily sample from a test subject and comparing this concentration with a reference for the concentration of the signature compound in an individual who does not suffer from colorectal cancer, wherein:
(i) an increase in the concentration of a signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, or
(ii) a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the subject is suffering from colorectal cancer, or has a pre-disposition thereto, or provides a negative prognosis of the subject's condition, wherein formulae (I), (II) and (III) are:
R¹-L¹-OH (I)
R²SR³ (II)
R⁴-L²-L³-OH (III),
wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

2. A method for determining the efficacy of treating a subject suffering from colorectal cancer with a therapeutic agent or a specialised diet, the method comprising analysing the concentration of a signature compound in a bodily sample from a test subject and comparing this concentration with a reference for the concentration of the signature compound in a sample taken from the subject at an earlier time point, wherein:
(i) a decrease in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or (ii) an increase in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the treatment regime with the therapeutic agent or the specialised diet is effective, or wherein (i) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or (ii) a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, suggests that the treatment regime with the therapeutic agent or the specialised diet is ineffective, wherein formulae (I), (II) and (III) are:
R¹-L¹-OH (I)
R²SR³ (II)
R⁴-L²-L³-OH (III),
wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

3. The method according to either claim 1 or 2, wherein the signature compound is a C₁-C₁₂ ester, a C₃₋₈ ester, or a C₅₋₆ ester.

4. The method according to any preceding claim, wherein where the ester is an ester of formula IV:
R⁶C(O)OR⁷ (IV),
R⁶ and R⁷ are independently a C₁₋₆ alkyl, a C₂₋₆ alkenyl or a C₂₋₆ alkynyl, or wherein R⁶ and R⁷ are independently a C₁₋₄ alkyl, a C₂₋₄ alkenyl or a C₂₋₄ alkynyl, and optionally wherein R⁶ and R⁷ are independently a C₁₋₃ alkyl, a C₂₋₃ alkenyl or a C₂₋₃ alkynyl.

5. The method according to claim 4, wherein R⁶ is methyl, ethyl or 1-propynyl; and/or R⁷ is methyl, n-propanyl or 2-propenyl.

6. The method according to any preceding claim, wherein the C₁-C₁₂ ester is propyl propionate, allyl acetate or methyl 2-butynoate.

7. The method according to any preceding claim, wherein the signature compound is a C₃₋₂₀ cycloalkane, or a C₃₋₂₀ cycloalkene, or C₃₋₁₅ cycloalkane or a C₃₋₁₅ cycloalkene, or a C₃₋₁₀ cycloalkane or a C₃₋₁₀ cycloalkene, or a C₅₋₁₀ cycloalkene, or a C₈₋₁₀ cycloalkene.

8. The method according to any preceding claim, wherein the C₃₋₂₀ cycloalkane or C₃₋₂₀ cycloalkene is cyclopropane, or 3,4-dimethyl-1,5-cyclooctadiene.

9. The method according to any preceding claim, wherein the signature compound is a C₁₋₂₀ alkane, a C₂₋₂₀ alkene, or a C₂₋₂₀ alkyne, preferably wherein the compound is a C₄₋₁₂ alkane, a C₄₋₁₂ alkene or a C₄₋₁₂ alkyne, or a C₆₋₁₀ alkane, a C₆₋₁₀ alkene or a C₆₋₁₀ alkyne, or a C₇₋₉ alkane, a C₇₋₉ alkene or a C₇₋₉ alkyne, or a C₈ alkane.

10. The method according to any preceding claim, wherein the C₁₋₂₀ alkane, C₂₋₂₀ alkene, or C₂₋₂₀ alkyne is 3-ethyl-hexane.

11. The method according to any preceding claim, wherein when the signature compound is an alcohol of formula I:
R¹-L¹-OH (I),
R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl; and
L¹ is absent or a C₁₋₆ alkylene, a C₂₋₆ alkenylene or a C₂₋₆ alkynylene.

12. The method according to claim 11, wherein L¹ is absent or a C₁₋₃ alkylene, a C₂₋₃ alkenylene or a C₂₋₃ alkynylene, optionally wherein L¹ is absent or methylene.

13. The method according to any preceding claim, wherein R¹ is a C₃₋₁₂ cycloalkyl or a 3 to 12 membered heterocycle, optionally wherein R¹ is a C₅₋₆ cycloalkyl or a 5 to 6 membered heterocycle.

14. The method according to any preceding claim, wherein R¹ is a 5 membered heterocycle, preferably wherein R¹ is 1,3-dioxolanyl.

15. The method according to any preceding claim, wherein L¹ is absent and R¹ is a C₃₋₁₈ alkyl, a C₃₋₁₈ alkenyl or a C₃₋₁₈ alkynyl, optionally wherein R¹ is a C₆₋₁₀ alkyl, a C₆₋₁₂ alkenyl or a C₆₋₁₀ alkynyl, or a C₇₋₉ alkyl, a C₆₋₉ alkenyl or a C₆₋₉ alkynyl, and preferably wherein R¹ is 2,2,4-trimethyl-3-pentanyl.

16. The method according to any preceding claim, wherein the alcohol of formula (I) is 1,3-dioxolane-2-methanol or 2,2,4-trimethyl-3-pentanol.

17. The method according to any preceding claim, wherein when the signature compound is an alcohol of formula III:
R⁴-L²-L³-OH (III),
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

18. The method according to any preceding claim, wherein L² is absent or O.

19. The method according to any preceding claim, wherein L³ is absent or a C₁₋₃ alkylene, a C₂₋₃ alkenylene or a C₂₋₃ alkynylene, optionally wherein L³ is absent, methylene or ethylene, or wherein L³ is absent or ethylene.

20. The method according to any preceding claim, wherein R⁴ is a C₆₋₁₂ aryl or a 5 to 12 membered heteroaryl, optionally wherein R⁴ is a phenyl or a 5 to 6 membered heteroaryl.

21. The method according to any preceding claim, wherein L² and L³ are absent and R³ is a C₃₋₁₈ alkyl, a C₃₋₁₈ alkenyl or a C₃₋₁₈ alkynyl, or wherein R³ is a C₅₋₁₇ alkyl, a C₅₋₁₇ alkenyl or a C₅₋₁₇ alkynyl, or wherein R³ is 1-undecanyl.

22. The method according to any preceding claim, wherein the alcohol of formula (III) is 2-phenoxy-ethanol, 1-undecanol or phenol, and preferably phenol.

23. The method according to any preceding claim, wherein when the signature compound is a sulphide of formula (II):
R²SR³ (II),
R² and R³ are independently a C₁₋₆ alkyl, a C₂₋₆ alkenyl or a C₂₋₆ alkynyl.

24. The method according to any preceding claim, wherein R² and R³ are independently a C₁₋₃ alkyl, a C₂₋₃ alkenyl or a C₂₋₃ alkynyl, or wherein R² and R³ are both methyl.

25. The method according to any preceding claim, wherein the sulphide is dimethyl sulphide.

26. The method according to any preceding claim, wherein the signature compound is a volatile organic compound (VOC).

27. The method according to any preceding claim, wherein the bodily sample is a breath sample from the test subject.

28. The method according to any preceding claim, wherein the sample is collected by the subject performing exhalation through the mouth and/or nose, preferably after nasal inhalation.

29. The method according to any preceding claim, wherein the signature compound is selected from a group consisting of: propyl propionate, allyl acetate, methyl 2-butynoate, 1,3-Dioxolane-2-methanol, 2,2,4-Trimethyl-3-pentanol, cyclopropane, 3,4-dimethyl-1,5-Cyclooctadiene, dimethyl sulphide, 2-Phenoxy-ethanol, 1-undecanol, phenol, and 3-ethyl-hexane, or an analogue or derivative thereof.

30. An apparatus for diagnosing a subject suffering from colorectal cancer, or a pre-disposition thereto, or for providing a prognosis of the subject's condition, the apparatus comprising:—
(i) means for determining the concentration of a signature compound in a sample from a test subject; and
(ii) a reference for the concentration of the signature compound in a sample from an individual who does not suffer from colorectal cancer,
wherein the apparatus is used to identify: (i) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, or (ii) a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, thereby suggesting that the subject suffers from colorectal cancer, or has a pre-disposition thereto, or provides a negative prognosis of the subject's condition, wherein formulae (I), (II) and (III) are:
R¹-L¹-OH (I)
R²SR³ (II)
R⁴-L²-L³-OH (III),
wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
so L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

31. An apparatus for determining the efficacy of treating a subject suffering from colorectal cancer with a therapeutic agent or a specialised diet, the apparatus comprising:—
(a) means for determining the concentration of a signature compound in a sample from a test subject; and
(b) a reference for the concentration of the signature compound in a sample taken from the subject at an earlier time point,
wherein the apparatus is used to identify:
(i) a decrease in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or an increase in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, thereby suggesting that the treatment regime with the therapeutic agent or the specialised diet is effective; or
(ii) an increase in the concentration of the signature compound selected from a C₁₋₁₂ ester, a C₃₋₂₀ cycloalkane, a C₃₋₂₀ cycloalkene, an alcohol of formula (I), a sulphide of formula (II), or an analogue or derivative thereof, in the bodily sample from the test subject, compared to the reference, or a decrease in the concentration of the signature compound selected from a C₁₋₂₀ alkane, a C₂₋₂₀ alkene, a C₂₋₂₀ alkyne, and an alcohol of formula (III), or an analogue or derivative thereof, in the bodily sample from the test subject, compared to the reference, thereby suggesting that the treatment regime with the therapeutic agent or the specialised diet is ineffective, wherein formulae (I), (II) and (III) are:
R¹-L¹-OH (I)
R²SR³ (II)
R⁴-L²-L³-OH (III),
wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

32. An apparatus according to either claim 30 or 31, wherein the signature compound is as defined in any one of claims 3-29.

33. Use of a signature compound selected from the group consisting of a C₁₋₁₂ ester, a C₃₋₂₀ cycloalkane, a C₃₋₂₀ cycloalkene, a C₁₋₂₀ alkane, a C₂₋₂₀ alkene, a C₂₋₂₀ alkyne, an alcohol of formula (I), a sulphide of formula (II), and an alcohol of formula (III), or an analogue or derivative thereof, as a biomarker for diagnosing a subject suffering from colorectal cancer, or a pre-disposition thereto, or for providing a prognosis of the subject's condition, wherein formulae (I), (II) and (III) are:
R¹-L-OH (I)
R²SR³ (II)
R⁴-L²-L³-OH (III),
wherein R¹ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L¹ is absent or a C₁₋₆ alkylene, a C₂₋₆ alkenylene or a C₂₋₆ alkynylene;
R² and R³ are independently a C₁₋₆ alkyl, a C₂₋₆ alkenyl or a C₂₋₆ alkynyl;
R⁴ is a C₁₋₂₀ alkyl, a C₂₋₂₀ alkenyl, a C₂₋₂₀ alkynyl, a C₃₋₁₂ cycloalkyl, a C₆₋₁₂ aryl, a 3 to 12 membered heterocycle or a 5 to 12 membered heteroaryl;
L² is absent or O, S or NR⁵;
L³ is absent or a C₁₋₆ alkylene, a C₂₋₆ alkenylene or a C₂₋₆ alkynylene; and
R⁵ is H or a C₁₋₆ alkyl, a C₂₋₆ alkenyl or a C₂₋₆ alkynyl.

34. Use according to claim 33, wherein the signature compound is as defined in any one of claims 3-29.
